(19) United States
(12) Patent Application Publication
(10) Pub. No.: US 2013/0177980 A1
(43) Pub. Date: Jul. 11, 2013

(54) SYNTHETIC MATRICES FOR SELF-RENEWAL AND EXPANSION OF STEM CELLS

(71) Applicants: Shyni Varghese, San Diego, CA (US); Chien-Wen Chang, San Diego, CA (US); Yongsung Hwang, Chula Vista, CA (US)

(72) Inventors: Shyni Varghese, San Diego, CA (US); Chien-Wen Chang, San Diego, CA (US); Yongsung Hwang, Chula Vista, CA (US)

(73) Assignee: THE REGENTS OF THE UNIVERSITY OF CALIFORNIA, Oakland, CA (US)

(21) Appl. No.: 13/706,900

(22) Filed: Dec. 6, 2012

Related U.S. Application Data
(60) Provisional application No. 61/567,588, filed on Dec. 6, 2011.

(51) Int. Cl. C12N 5/071; C08F 228/02; C08F 222/38; C12N 5/0735

(52) U.S. Cl. CPC C12N 5/0696; C12N 5/0606; C08F 228/02; C08F 222/38
USPC 435/366; 526/287; 526/289; 526/304

(57) ABSTRACT

Provided herein is a synthetic polymer-based hydrogel for the self-renewal and expansion of human stem cells such as embryonic stem cells (ESCs) and induced pluripotent stem cells (iPSCs). Also provided are methods of making and using the same.

### CROSS REFERENCE TO RELATED APPLICATION(S)

This application claims the benefit of priority under 35 U.S.C. §119(e) of U.S. Ser. No. 61/567,588, filed Dec. 6, 2011, the entire content of which is incorporated herein by reference.

### BACKGROUND OF THE INVENTION

1. Field of the Invention

The invention relates generally to hydrogels and more particularly to synthetic hydrogels for self-renewal and expansion of stem cells.

2. Background Information

Past research has shown that human pluripotent stem cells (hPSCs) such as human embryonic stem cells (hESCs) and induced pluripotent stem cells (iPSCs) grow best when cultured on feeder cells such as mouse embryonic fibroblasts or extra-cellular matrix (ECM)-rich Matrigel. Since the isolation of hESCs, there has been tremendous interest in developing defined, scalable in vitro culture conditions that can support their growth. These efforts have led to the development of multiple defined growth media, but these still require either feeder layers such as mouse embryonic fibroblasts (MEF) or biologically derived matrices such as Matrigel for maintenance of pluripotency and self-renewal of hPSCs.

Development of chemically defined matrices is a challenging task because of the myriad of physicochemical signals that MEFs and Matrigel provide. Within these limitations, recent advances in the field of biomaterials have led to identification of substrates—both naturally derived and synthetic—for the self-renewal of hPSCs. High throughput screening technologies have contributed significantly towards the development of these chemically defined culture conditions.

Thus, developing cost-effective, and scalable synthetic matrices for long-term expansion of human pluripotent stem cells (hPSCs) is important to realize the potential of these cells, ranging from drug screening platforms to regenerative medicine. Additionally, such synthetic matrices will be ideal tools to understand the molecular mechanisms that control fate and commitment of hPSCs.

### SUMMARY OF THE INVENTION

The present invention is based on the finding that synthetic hydrogels can be used for self-renewal and expansion of stem cells. Accordingly, in one aspect, the invention provides a synthetic hydrogel that includes a heparin mimetic moiety and an acrylamide monomer. In various embodiments, the heparin mimetic moiety may be sodium-4-styrenesulfonate (SS) or poly(sodium-4-styrenesulfonate) (PSS). In certain embodiments, the molar fraction of PSS ranges from about 0.5 to 2. In certain embodiments, the PSS has a matrix rigidity of about 54 kPa, about 138 kPa, or about 344 kPa. In other embodiments, the molar ratio of the acrylamide monomer to PSS is 6:0.5, 6:1, or 6:2. In yet other embodiments, the molar ratio of the acrylamide monomer to PSS is 6:2. In another embodiment, the hydrogel further includes a bisacrylamide.

In another aspect, the invention provides a method for synthesizing a synthetic hydrogel. The method includes copolymerizing an acrylamide monomer with an ionic monomer. In various embodiments, the heparin mimetic moiety may be sodium-4-styrenesulfonate (SS) or poly(sodium-4-styrenesulfonate) (PSS). In certain embodiments, the molar fraction of PSS ranges from about 0.5 to 2. In certain embodiments, the PSS has a matrix rigidity of about 54 kPa, about 138 kPa, or about 344 kPa. In other embodiments, the molar ratio of the acrylamide monomer to PSS is 6:0.5, 6:1, or 6:2. In yet other embodiments, the molar ratio of the acrylamide monomer to PSS is 6:2. In another embodiment, the hydrogel further includes a bisacrylamide.

In another aspect, the invention provides a method for synthesizing a synthetic hydrogel. The method includes dissolving an acrylamide monomer and an ionic monomer in deionized water, and polymerizing the acrylamide monomer and ionic monomer using a crosslinker and a redox initiator. In various embodiments, the heparin mimetic moiety may be sodium-4-styrenesulfonate (SS) or poly(sodium-4-styrenesulfonate) (PSS). In certain embodiments, the molar fraction of PSS ranges from about 0.5 to 2. In certain embodiments, the PSS has a matrix rigidity of about 54 kPa, about 138 kPa, or about 344 kPa. In other embodiments, the molar ratio of the acrylamide monomer to PSS is 6:0.5, 6:1, or 6:2. In yet other embodiments, the molar ratio of the acrylamide monomer to PSS is 6:2. In another embodiment, the hydrogel further includes a bisacrylamide.

In yet another aspect, the invention provides a method for expanding stem cells. The method includes seeding a stem cell onto a synthetic hydrogel comprising a heparin mimetic moiety and an acrylamide monomer. In various embodiments, the heparin mimetic moiety may be sodium-4-styrenesulfonate (SS) or poly(sodium-4-styrenesulfonate) (PSS). In certain embodiments, the molar fraction of PSS ranges from about 0.5 to 2. In certain embodiments, the PSS has a matrix rigidity of about 54 kPa, about 138 kPa, or about 344 kPa. In other embodiments, the molar ratio of the acrylamide monomer to PSS is 6:0.5, 6:1, or 6:2. In yet other embodiments, the molar ratio of the acrylamide monomer to PSS is 6:2. In another embodiment, the hydrogel further includes a bisacrylamide. In yet other embodiments, the stem cell is selected from the group consisting of HUES9, HUES9-Oct4-GFP, HUES6, and human induced pluripotent stem cells (hiPSCs).

### BRIEF DESCRIPTION OF THE DRAWINGS

FIG. 1 is a pictorial and graphical diagram showing that PSS-based hydrogels support growth of HUES9 cells in vitro. FIGS. 1A and 1B show a schematic of PSS-based hydrogel(s) synthesized by copolymerizing acrylamide with sodium 4-vinylbenzenesulfonate with bisacrylamide as a crosslinking agent. FIG. 1C shows representative phase contrast images of HUES9 colonies on PAm₆-co-PSS₂ hydrogel after 7 days in culture in StemPro medium. FIG. 1D shows phase contrast images of HUES9 colonies on Matrigel after 7 days in culture in StemPro medium. FIG. 1E shows phase contrast images of HUES9 colonies on mouse embryonic fibroblasts (MEFs) after 7 days in culture in StemPro medium. FIG. 1F shows the results of population doubling of HUES9 cells over five days under the conditions in FIGS. 1C-1E. Scale bar: 200 μm. Values are shown as mean±SD. ***p<0.001.

FIG. 2 is a pictorial and graphical and pictorial diagram showing that PSS-based synthetic matrices supported long-term maintenance of HUES9 in StemPro medium. FIG. 2A shows images of HUES9 cells grown on PAm₆-co-PSS₂ hydrogels in StemPro medium for 20 passages stained positive for NANOG and OCT4. The nuclei are stained blue with Hoescht 33342. The inset shows higher magnification images. Scale bar: 200 μm (main images) and 100 μm (inset images). FIG. 2B shows the results of quantitative PCR of HUES9 cells grown on PAm₆-co-PSS₂ hydrogels, which show similar expression levels of OCT4 and NANOG to that of Matrigel. FIG. 2C shows representative FACS profiles of HUES9 cells grown on PAm₆-co-PSS₂ hydrogel and Matrigel, which again shows that PAm₆-co-PSS₂ hydrogels can support in vitro self-renewal of HUES9 cells similar to Matrigel.

FIG. 3 is a pictorial and graphical diagram showing in vitro differentiation of HUES9 cells passaged 20 times using PAm₆-co-PSS₂ hydrogels. FIG. 3A shows that HUES9 cells grown on PAm₆-co-PSS₂ hydrogels formed embryoid bodies (EBs). FIG. 3B show the results of immunofluorescence staining showing differentiation of these cells into endoderm (SOX17), mesoderm (SMA), and ectoderm (Nestin) lineages. Scale bar=100 μm. FIG. 3C shows quantitative PCR results for differentiated HUES9 cells showing expression of ectoderm (CXCR4, FOXA2, SOX17), mesoderm (SMA, ACTC1), and ectoderm markers (Nestin, SOX1, SOX2). FIG. 3D shows the results from karyotype analysis of HUES9 cells grown on PAm6-co-PSS2 hydrogel showing a normal euploid karyotype. Values are shown as mean±SD. **p<0.01 and ***p<0.001.

FIGS. 4A and 4C show that PAm₆-co-PSS₂ hydrogel supported in vitro growth of HUES6 grown over multiple passages in StemPro medium (images taken after passage 7). FIGS. 4B and 4D show that PAm₆-co-PSS₂ hydrogel supported in vitro growth of human induced pluripotent stem cells (hiPSCs) grown over multiple passages in StemPro medium (images taken after passage 9). Immunofluorescence shows the presence of NANOG and OCT4, while nuclei are stained with Hoescht 33342. Scale bar: 200 μm (main images) and 100 μm (inset images).

FIG. 5 is a pictorial diagram showing the effect of matrix rigidity, chemistry, and hydrophilicity on hPSCs. FIG. 5A shows representative phase contrast images of adhered HUES9-Oct4-GFP cells on PSS-based hydrogels with varying bulk rigidity (top) and their corresponding fluorescence images (bottom). FIG. 5B shows images of HUES9 cell growth on PAm₆-co-PSS₂ hydrogels having higher elastic modulus (about 344 kPa). FIG. 5C shows images of HUES9-Oct4-GFP cells on PSS-based hydrogels with varying mole ratio of acrylamide to sodium 4-vinylbenzene sulfonate (Am:SS; 6:0.5, 6:1, and 6:2); PAm₆-co-PSS_{0.5}, PAm₆-co-PSS₁, PAm₆-co-PSS₂ (top). FIG. 5D shows representative phase contrast images of hPSCs grown on hydrogels containing different chemistries and hydrophilicities while maintaining identical sulfate functional groups and matrix rigidities. Scale bar: 200 μm.

FIG. 6A shows the effect of hydrogel composition on adhesion and growth of hPSCs by varying the amount of PSS content within the hydrogels (Am:PSS 6:0.5, 6:1, 6:2). FIGS. 6B and 6C show that unlike PAm₆-co-PSS₂, HUES9 cells exhibited minimal to no cell adhesion on PAm₆-co-PA2AGA₂ hydrogels.

FIG. 7 shows a comparison of the physicochemical characteristics (elastic modulus, hydrophilicity, and topography) of copolymer hydrogels bearing carboxylate groups (PAm₆-co-PA2AGA₂) that were synthesized having similar characteristics to that of PAm₆-co-PSS₂. FIG. 7A shows the scan area of PAm₆-co-PA2AGA₂ and PAm₆-co-PSS₂ copolymer hydrogels. FIG. 7B shows the physical properties (Panel 1), functionality (Panel 2), and cell adhesion (Panel 3) characteristics of PAm₆-co-PA2AGA₂ and PAm₆-co-PSS₂.

FIG. 8 is a pictorial and graphical diagram showing characterization of the cell-material interface. FIG. 8A shows the results of quantification of various extracellular matrix proteins adsorbed onto PAm₆-co-PSS₂ hydrogel and PAm₆-co-PA2AGA₂ hydrogel. BSA=bovine serum albumin; VN=vitronectin; Col1=collagen type I; Col4=collagen type IV; LN=laminin; FN=fibronectin. FIG. 8B shows the results of bFGF adsorption onto PAm₆-co-PSS₂ and PAm₆-co-PA2AGA₂ hydrogels. Values are shown as mean±SD. *p<0.05 and **p<0.01. FIGS. 8C and 8D show results of hierarchical cluster analysis of the transcription profile of HUES9 cells cultured on MEFs, PAm₆-co-PSS₂, and PAm₆-co-PSPA₂. Expression levels are normalized to that of Matrigel. The notations *, **, #, and ## indicate 2-5 times, 5-10 times, 10-15 times, and >15 times of relative fold inductions, respectively.

FIG. 9 is a pictorial diagram showing the variation of water contact angle (WCA).

### DETAILED DESCRIPTION OF THE INVENTION

The present invention is based on the finding that synthetic hydrogels can be used for self-renewal and expansion of stem cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Standard techniques for growing cells, separating cells, analyzing gene expression, determining cell surface biomarkers and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described by Freshney, R. I., Culture of Animal Cells: A Manual of Basic Technique, 5e. 2007, John Wiley & Sons, Inc., New Jersey Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “human Pluripotent Stem Cells” or “hPSCs,” of which “human Embryonic Stem Cells” or hESCs and “human induced pluripotent stem cells” or hiPSCs are a subset, refers to cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm and ectoderm). The term includes both established lines of stem cells of various kinds, and cells obtained from primary tissue that are pluripotent in the manner described. Included in the definition of pluripotent stem cells (PSCs) are embryonic cells of various types, especially including human embryonic stem cells (hESCs), described by Thomson et al. (Science 282: 1145, 1998). Other types of pluripotent cells are also included in the term. Human Pluripotent Stem Cells includes stem cells which may be obtained from human umbilical cord or placental blood as well as human placental tissue. Any cells of primate origin that are capable of producing progeny that are derivatives of all three germinal layers are included, regardless of whether they were derived from embryonic tissue, fetal, or other sources.

An “induced pluripotent stem cell” refers to a pluripotent stem cell artificially (e.g. non-naturally, in a laboratory setting) derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development.

Pluripotent stem cell (PSC) cultures are described as “undifferentiated” when a substantial proportion of stem cells and their derivatives in the population display morphological characteristics of undifferentiated cells, clearly distinguishing them from differentiated cells of embryo or adult origin. Undifferentiated PSCs are easily recognized by those skilled in the art, and typically appear in the two dimensions of a microscopic view in colonies of cells with high nuclear/cytoplasmic ratios and prominent nucleoli. It is understood that colonies of undifferentiated cells in the population will often be surrounded by neighboring cells that are differentiated.

Pluripotent stem cells may express one or more of the stage-specific embryonic antigens (SSEA) 3 and 4, and markers detectable using antibodies designated Tra-1-60 and Tra-1-81 (Thomson et al., Science 282:1145, 1998). Differentiation of pluripotent stem cells in vitro is known to result in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde, and then developing with Vector Red as a substrate, as described by the manufacturer (Vector Laboratories, Burlingame Calif.). Undifferentiated pluripotent stem cells also typically express Oct-4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of pluripotent stem cells can be confirmed, for example, by injecting cells into severe combined immunodeficient (SCID) mice, fixing the teratomas that form using 4% paraformaldehyde, and then examining them histologically for evidence of cell types from the three germ layers. Alternatively, pluripotency may be determined by the creation of embryoid bodies (EBs) and assessing the EBs for the presence of markers associated with the three germinal layers.

The term “embryonic stem cell” or “ESC” or “hESCs” refers to pluripotent cells, preferably of primates, including humans (hESCs), which are isolated from the blastocyst stage embryo. The following phenotypic markers are known to be expressed by human embryonic stem cells: SSEA-3, SSEA-4, TRA-1-60, TRA-1-81, CD9, alkaline phosphatase, Oct 4, Nanog, Rex 1, Sox2 and TERT. See Ginis, et al., Dev. Biol, 269(2), 360-380 (2004); Draper, et al., J. Anat., 200(Pt. 3), 249-258, (2002); Carpenter, et al., Cloning Stem Cells, 5(1), 79-88 (2003); Cooper, et al., J. Anat., 200(Pt. 3), 259-265 (2002); Oka, et al., Mol. Biol. Cell, 13(4), 1274-81 (2002); and Carpenter, et al., Dev. Dyn., 229(2), 243-258 (2004).

The term “confluence” refers to the density of cells grown in culture. A culture of cells which is 10% confluent, is used to describe a population of cells which covers approximately 10% of the surface area of the culture dish (flask) in which the cells are grown. Similarly, a culture of cells which is 90% confluent, is used to describe a population of cells which covers approximately 90% of the surface area of the culture dish (flask) in which the cells are grown. In the present invention, cells are generally grown to at least 50%, about 80-90+% confluence, about 90%, about 90+% confluence before passaging and being subjected to a differentiation step. If a cell culture is deemed confluent, the culture completely covers (approximately 100%) of the culture dish.

The term “differentiation” is used to describe a process wherein an unspecialized (“uncommitted”) or less specialized cell acquires the features of a more specialized cell such as, for example, human embryonic stem cell derived epithelial cell (hESC-EC), human embryonic stem cell derived mesenchymal cell (hESC-MC), or where a more specialized intermediate cell, such as a mesenchymal cell (hES-MC) or epithelial cell (hES-EC) becomes an even more specialized cell such as a bone cell, a cartilage cell or a smooth muscle cell. A differentiated or differentiation-induced cell is one that has taken on a more specialized (“committed”) position within the lineage of a cell. The term “committed,” when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” may be used to refer to the physical selection of one or more cells out of a group of two or more cells, wherein the cells are selected based on cell morphology and/or the expression of various markers. It is noted herein that in various aspects of the present invention, one of the principal benefits is that isolation of cells, because of the levels of confluence and population consistency, do not require a separate isolation technique or step. Within this context, the term “isolating” may simply refer to the passaging of cells without further isolation steps being used to provide unexpected consistency of the final isolated cell population.

As used herein, the term “express” refers to the transcription of a polynucleotide or translation of a polypeptide (including a marker) in a cell, such that levels of the molecule are measurably higher in or on (cell surface) a cell that expresses the molecule than they are in a cell that does not express the molecule. Methods to measure the expression of a molecule are well known to those of ordinary skill in the art, and include without limitation, Northern blotting, RT-PCR, in situ hybridization, Western blotting, and immunostaining.

As used herein, the term “markers” describe nucleic acid or polypeptide molecules that are differentially expressed in a cell of interest, in particular, human embryonic stem cells (hESCs). In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

The term “passaged” or “passaging” is used to describe the process of splitting cells and transferring them to a new cell vial for further growth/regrowth or for storage. The preferred adherent cells (or even embryoid bodies) according to the present invention may be passaged using enzymatic (trypsinase, Accutase™, collagenase) passage, manual passage (mechanical, with, example, a spatula or other soft mechanical utensil or device) and other non-enzymatic methods, such as cell dispersal buffer. Cells are generally grown to at least about 50% confluence, preferably about 75-90% confluence, preferably about 90% confluence. In most instances after reaching confluence, the cells are then isolated or passaged and further grown and/or differentiated.

The term “hydrogel” as used herein refers to three-dimensional hydrophilic polymeric networks. Hydrogels have high water content, providing an environment sufficient transportation of nutrients and waste products, which is essential for cell growth.

The term “growth factor” as used herein refers to a molecule that elicits a biological response to improve tissue regeneration, tissue growth and organ function. Exemplary growth factors include, but are not limited to morphogens such as basic Fibroblast growth factor (bFGF). bFGF is a critical component of conventional human embryonic stem cell culture medium; the growth factor is necessary for the cells to remain in an undifferentiated state.

Growing evidence suggests that heparin molecules play a role in maintaining self-renewal of hPSCs. Recent studies have shown the role of MEF-secreted heparin sulfate proteoglycans on self-renewal of hESCs. Harnessing the beneficial effect of heparin moieties on modulating self-renewal of hPSCs, synthetic matrices displaying heparin-binding peptides were developed to support long-term self-renewal of hPSCs. The role of heparin moieties on self-renewal of hPSCs is not surprising given that heparin molecules can bind to soluble bFGF molecules and modulate their bioactivity; bFGF is an important biomolecule required for maintenance of self-renewal of hPSCs in vitro. Additionally, heparin molecules have been shown to protect bFGF from denaturation and proteolytic degradation, thereby increasing their longevity and function.

Synthetic heparin mimics such as poly(sodium-4-styrenesulfonate) (PSS) have been recently shown to bind to soluble bFGF and regulate FGF signaling akin to heparin molecules. Based on these findings along with the known role of bFGF molecules on in vitro self-renewal of hPSCs, synthetic hydrogels containing PSS moieties were developed to support long-term culture of hPSCs while maintaining their pluripotency. As such, the role of various physicochemical cues of the matrix on self-renewal of hPSCs was determined. Such easy-to-synthesize and cost effective synthetic matrices would not only accelerate the translational potential of hPSCs, but also provide a platform to decipher the interplay between various physicochemical cues on self-renewal of hPSCs. Additionally, these matrices may help to identify the myriad of molecular and signaling pathways dictating stem cell fate and commitment.

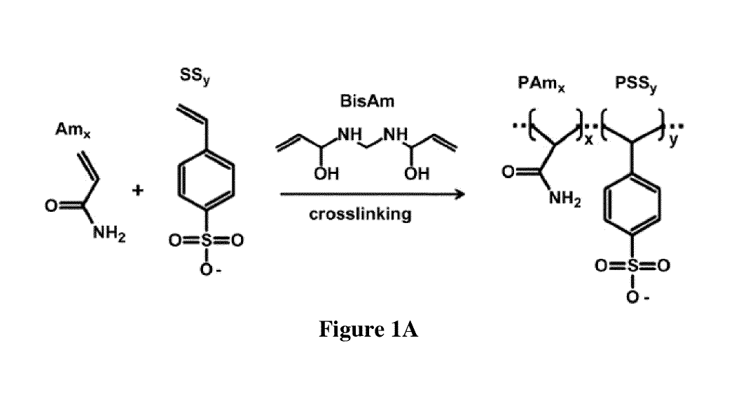

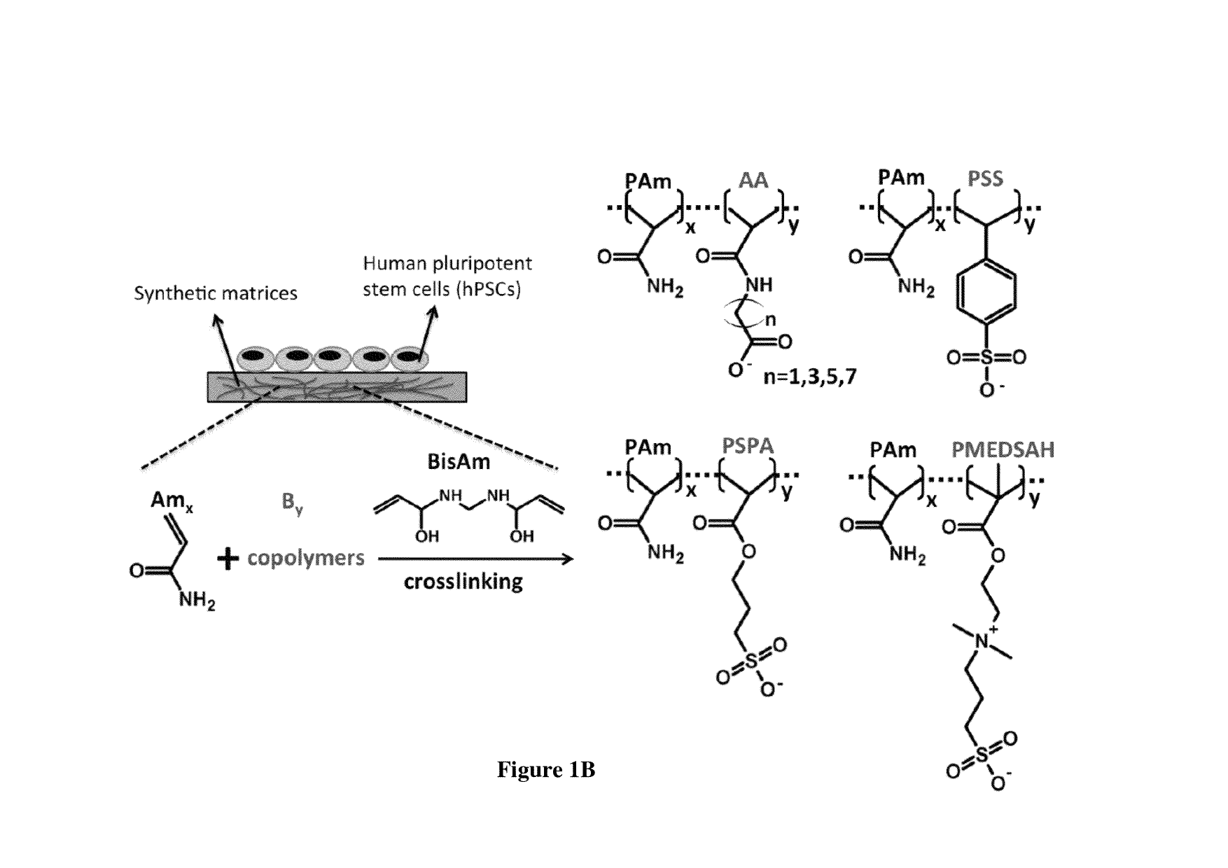

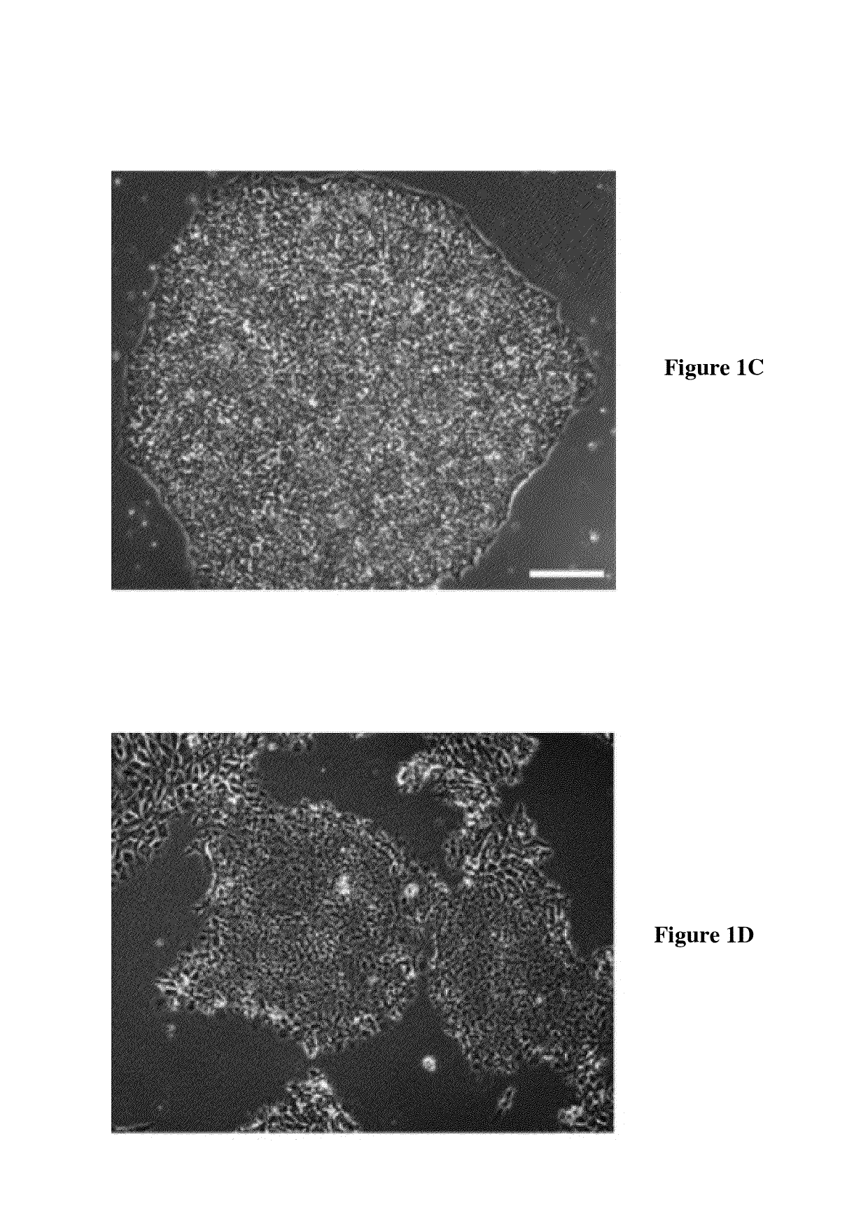

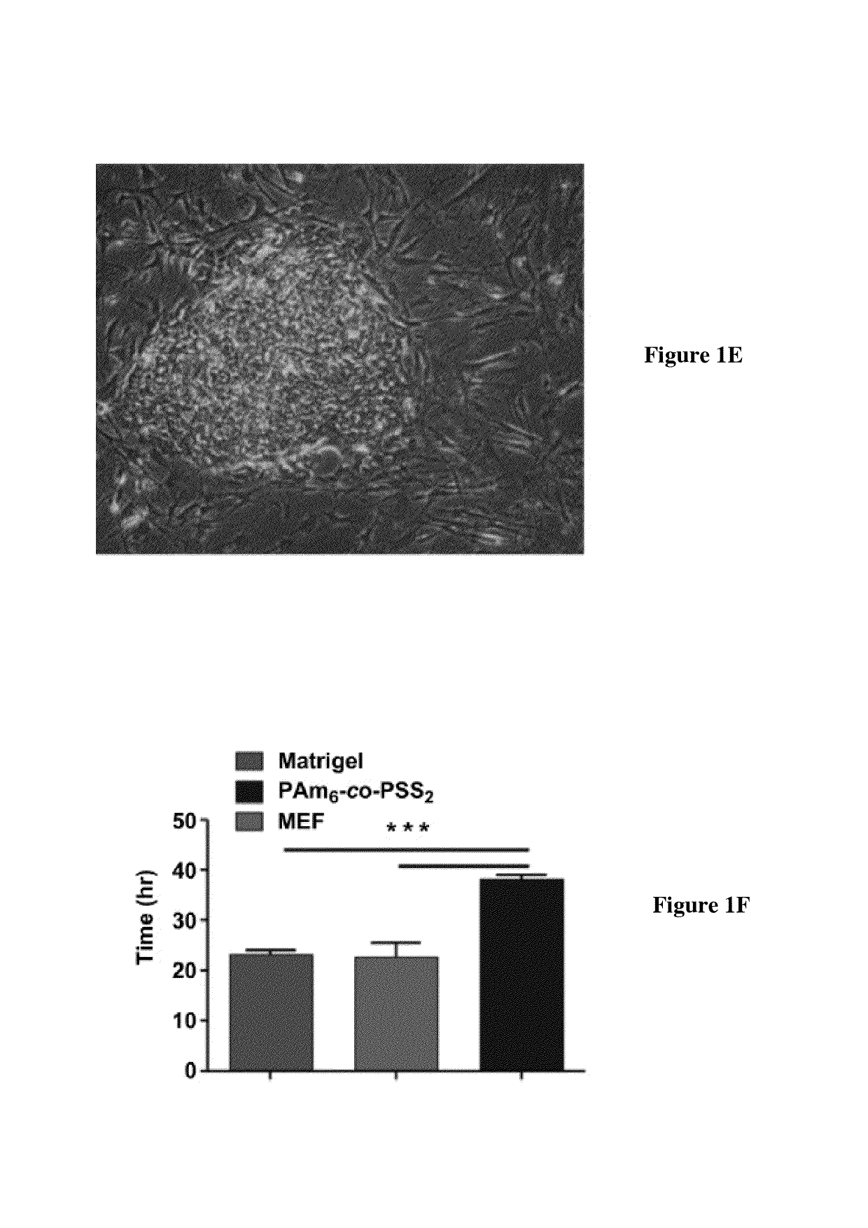

Accordingly, the present invention provides for the synthesis of hydrogels with varying elastic modulus, functional groups (e.g., SO₃H or COOH), matrix rigidity, charge density, and interfacial hydrophobicity by copolymerizing acrylamide (Am) with monomers containing either a sulfonate or a carboxylate functional group. Together, these hydrogels with varying physicochemical properties provide information on the effect of chemistry, functional group, rigidity, and hydrophilicity of the matrix on supporting self-renewal of hPSCs in vitro. The copolymer hydrogels are referred to as PAmₓ-co-PB_{y}, where PAm and PB represent the polymer components of the hydrogel, and x and y denote the mole ratio of the monomers used in hydrogel synthesis (see Table 1). For instance, the hydrogels synthesized by copolymerizing acrylamide (Am) and sodium 4-vinylbenzenesulfonate (SS) at a mole ratio of 6:2 is denoted as Pam₆-co-PSS₂ (FIG. 1A). FIG. 1B shows the schematic of the synthetic matrices containing PAm and PSS moieties (e.g., PAmₓ-co-PSS_{y}). The elastic modulus and hydrophilicity of these hydrogels are listed in Table 1, below. For example, the PAm₆-co-PSS₂ hydrogels exhibited an elastic modulus of 343.7±5.1 kPa, and a hydrophilicity of 23.0±2.0°.

Additional Pam₆-co-PSS₂ hydrogels were also synthesized with different bisacrylamide (Bis-AM, crosslinker) contents to vary the bulk rigidity. The Pam-co-PSS hydrogels with varying charge density were synthesized by copolymerizing Am monomers with varying molar fractions of PSS from about 0.5 to about 2. Together these hydrogels allow for the examination of the effect of functional groups, hydrophobicity, rigidity, and charge density of the hydrogel matrices on hPSC cellular responses.

As such, the present invention demonstrates the potential of synthetic hydrogels containing heparin-mimicking poly(styrene sulfonate) (PSS) moieties in supporting the in vitro growth and self-renewal of hPSCs. The synthetic matrix, PAm₆-co-PSS₂, supported the growth and expansion of multiple hPSC lines (HUES9, HUES6, and hiPSCs) through multiple passages (≧20 passages) while maintaining their pluripotency.

The results provided herein indicate that the presence of sulfonate groups alone is not sufficient to support self-renewal of hPSCs, but a combination of physical cues such as hydrophilicity and elastic modulus is required, thus exemplifying the delicate balance of insoluble and soluble cues of the niche on various cellular responses of hPSCs. Previous studies have shown that PMEDSAH-coated dishes having a water contact angle of about 17° supported self-renewal of hESCs in StemPro medium. However, PAm₆-co-PMEDSAH₂ hydrogels failed to support hPSCs adhesion and growth. Without being bound by theory, this could be attributed to the differences in chemical composition and/or the interfacial hydrophilicity of the matrix. The functional groups and interfacial hydrophilicity have been shown to play a key role in modulating protein adsorption and conformation of proteins, and sequestration of growth factors. These subtle changes of the cell-matrix can have a significant effect mediating the initial adhesion of hPSCs onto the matrix.

Despite having the same hydrophilicity, elastic modulus, and surface roughness, PAm₆-co-PSS₂ and PAm₆-co-PA2AGA₂ hydrogels elicited different cellular responses. Without being bound by theory, these differences in cellular responses could be attributed to changes in the ECM proteins that are adsorbed onto the hydrogels interface, with significant alterations in extent of BSA and VN adsorbed between the surfaces. Previous studies have shown that the adsorption of BSA and VN onto hydrogel surfaces can foster adhesion and self-renewal of hPSCs. It is also possible that besides the amount of proteins at the interface, the conformation of the proteins plays a role in mediating cell adhesion. Another possibility is the differences in matrix-bFGF binding strength, which could lead to changes in bFGF signaling.

PSS-based hydrogels support self-renewal and long-term expansion of hPSCs. The initial observation was that the human embryonic stem cells (hESCs, HUES9-Oct4-GFP and HUES9) grown on PAm₆-co-PSS₂ hydrogels adhered onto the underlying hydrogel and formed bright and compact colonies. However, differences in cell adhesion were observed between PAm₆-co-PSS₂ hydrogels and their control counterparts (i.e., MEF- and Matrigel-supported cultures). Observations after 24 hours of cell seeding indicated that the number of cells adhered onto both MEF and Matrigel were significantly higher compared to PAm₆-co-PSS₂ hydrogels. Despite these differences, the cells adhered onto PAm₆-co-PSS₂ hydrogels, proliferated and formed compact colonies similar to those observed on Matrigel and MEF-supported cultures (FIGS. 1C-1E). The population doubling of HUES9 cells on PAm₆-co-PSS₂ hydrogels was found to be about 38 hours, while that on MEF and Matrigel was found to be about 23 hours (FIG. 1F). However, without being bound by theory, the estimated population doubling time may be an overestimate given that the cell-cell adhesion of cells grown on the these hydrogels were significantly higher compared to those on Matrigel- and MEF-supported cultures; strong cell-cell adhesion limits the uniform dissociation of hESC colonies into single cells.

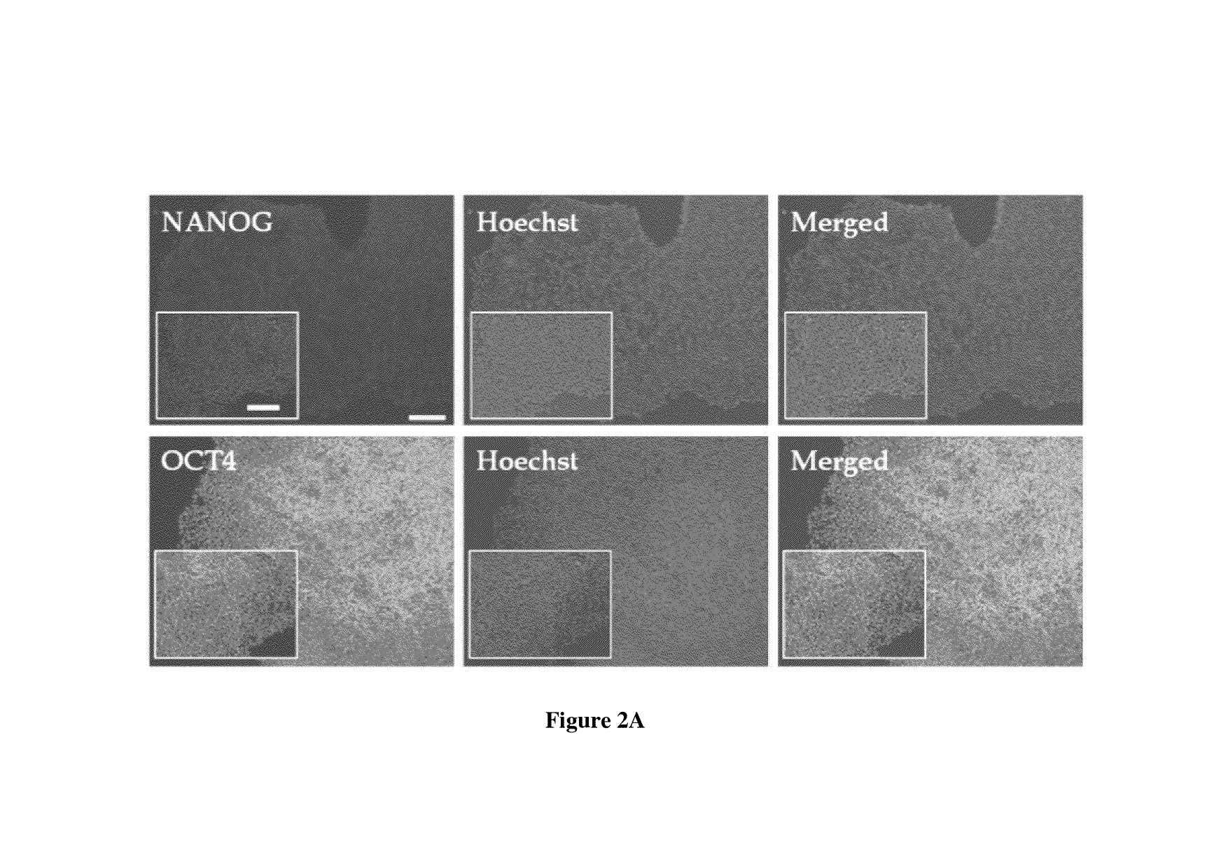

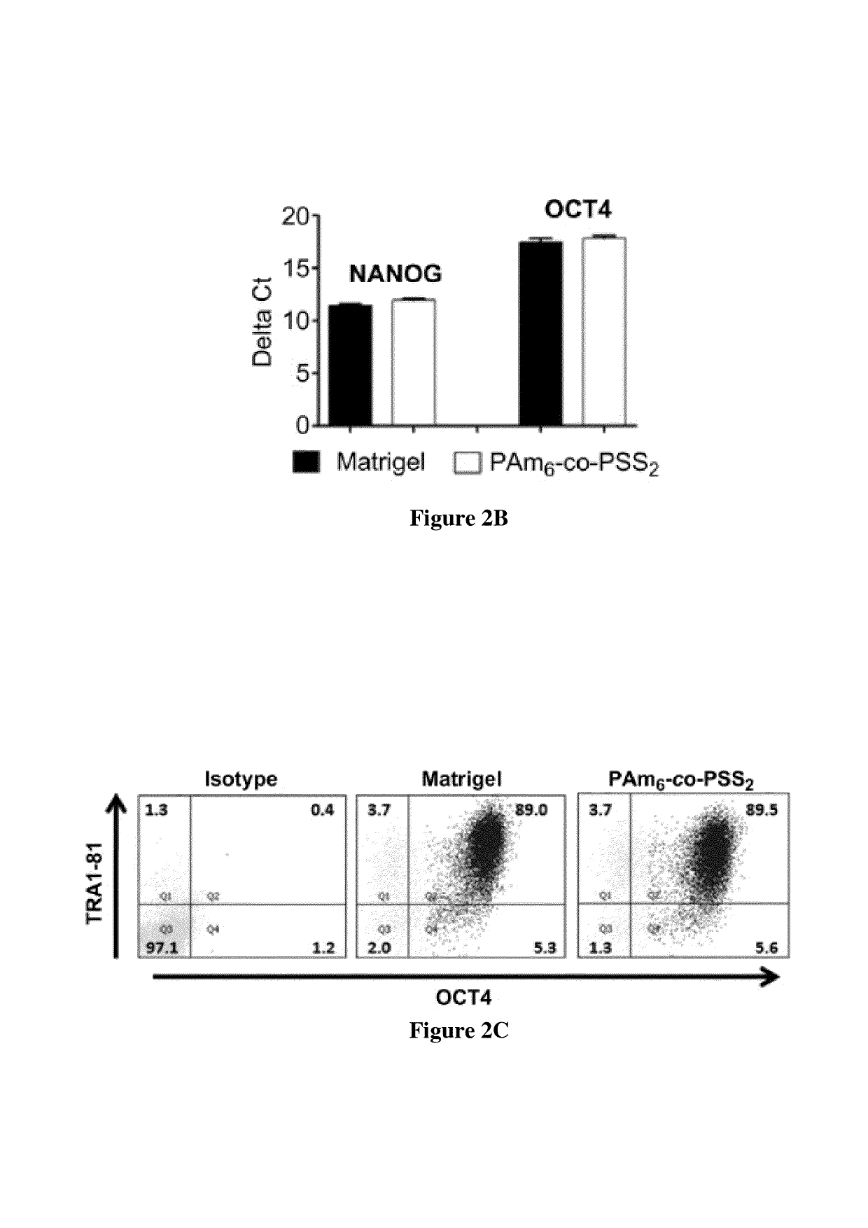

While these findings show that the HUES9 cells can adhere and grow on PAm₆-co-PSS₂ hydrogels, it was necessary to test whether the developed matrix could support the long-term expansion of hPSCs without compromising their pluripotency and karyotypic stability. The PAm₆-co-PSS₂ hydrogels indeed supported adhesion and long-term growth of HUES9 cells both in MEF-conditioned medium and chemically defined StemPro medium without affecting their pluripotency (FIG. 2A). HUES9 cells expanded on PAm₆-co-PSS₂ hydrogels with frequent splitting for over 20 passages (>8 months) using StemPro medium exhibited characteristic stem cell morphology and tight colony formation (FIG. 2A). The expanded HUES9 cells were positive for OCT4 and NANOG expression, compared to those cultured on Matrigel. The real-time PCR (qPCR) results indicate that hPSCs exhibit similar gene expression level of OCT4 and NANOG compared to those cultured on Matrigel (FIG. 2B). The pluripotency of HUES9 cells expanded on PAm₆-co-PSS₂ hydrogels was further confirmed by FACS analysis, which revealed a similar percentage of pluripotent cells between those cultured on PAm₆-co-PSS₂ and Matrigel, as evidenced by the population of OCT4 and TRA-1-81 positive cells (FIG. 2C).

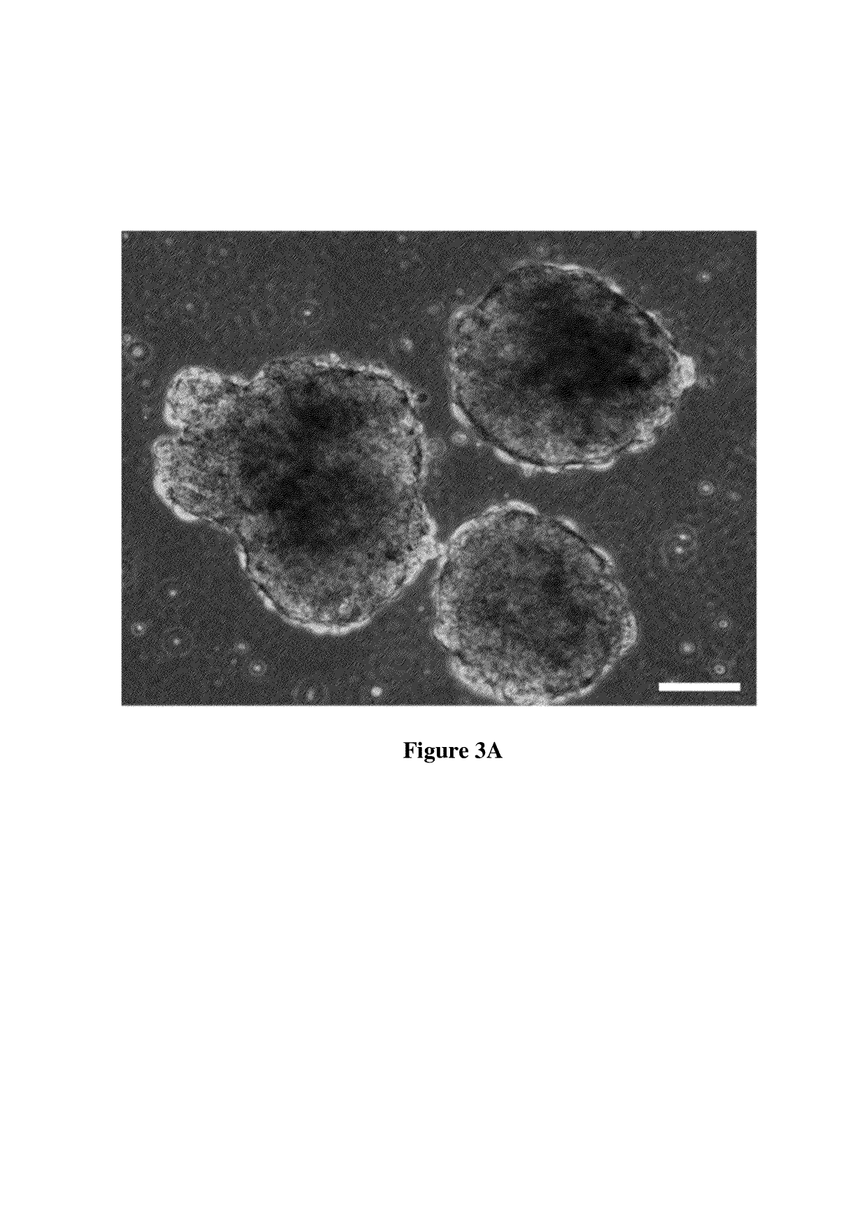

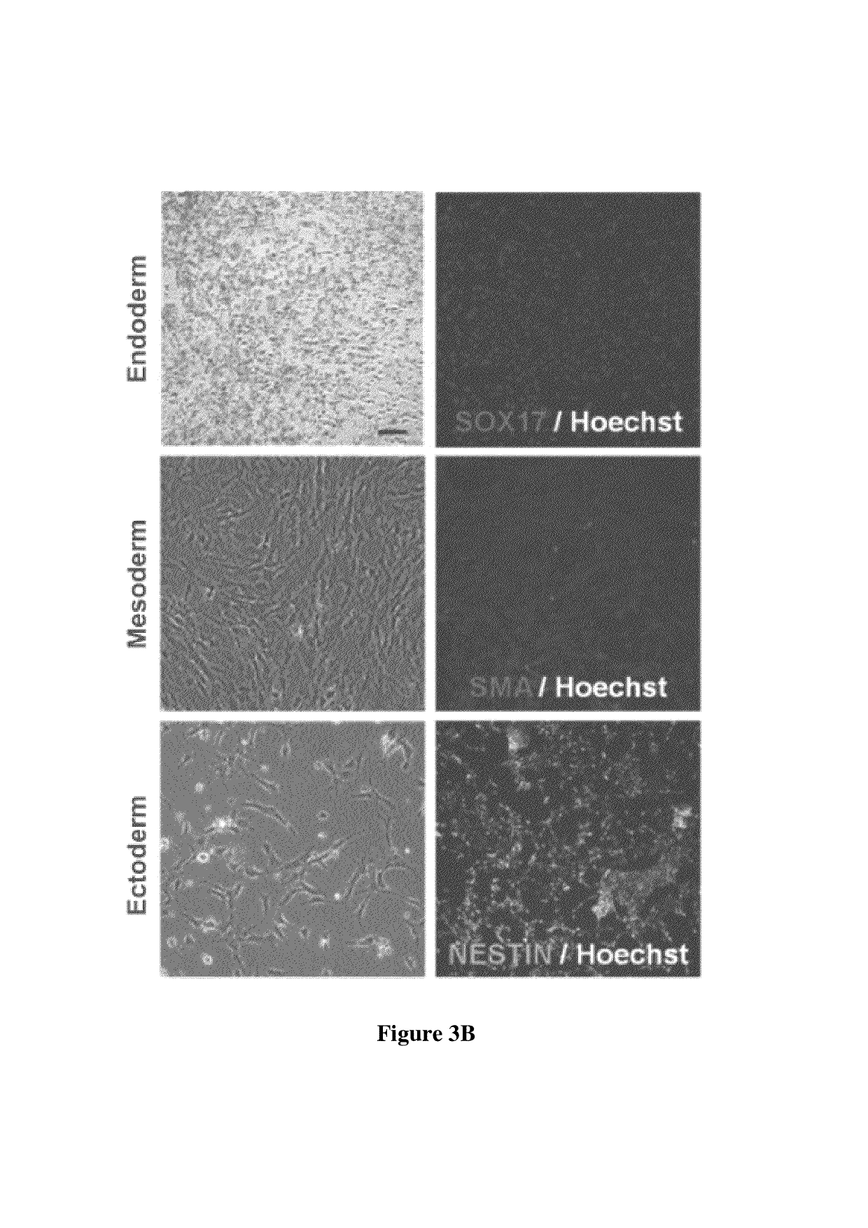

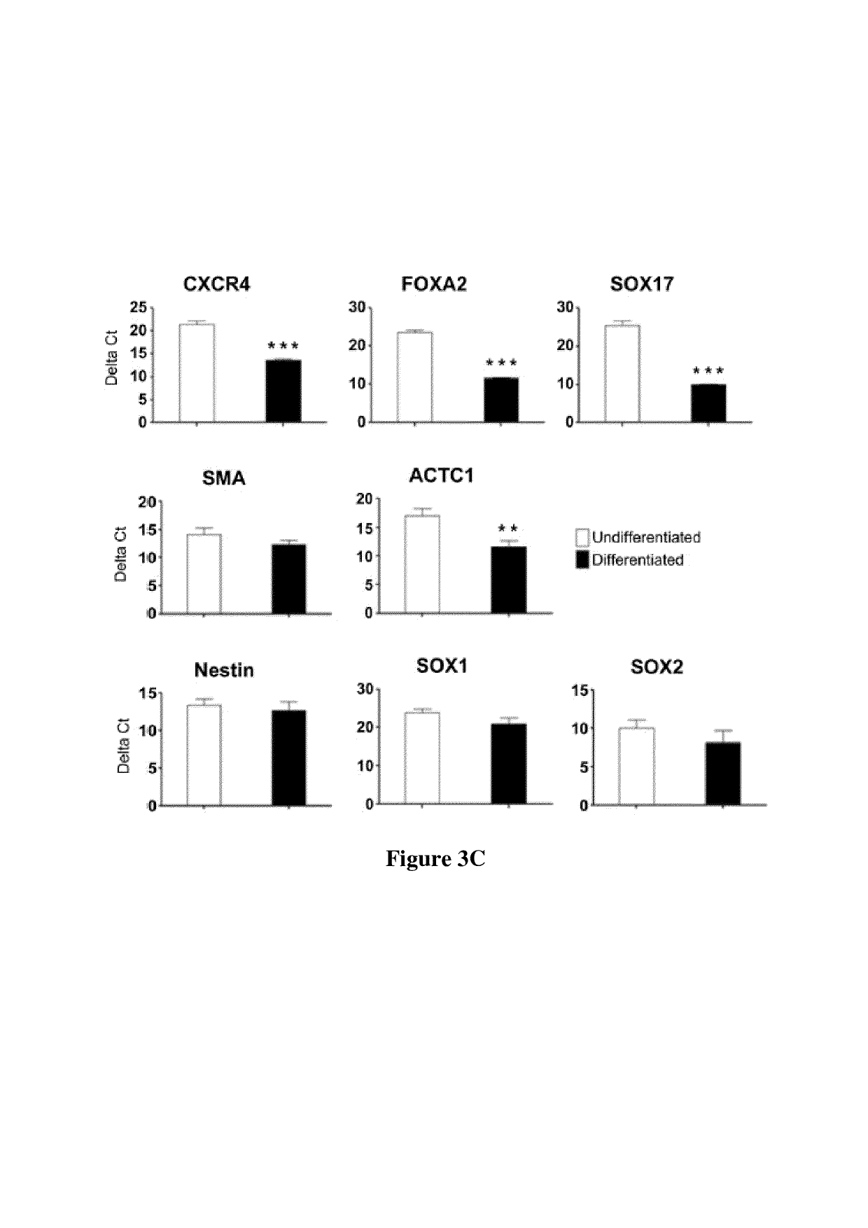

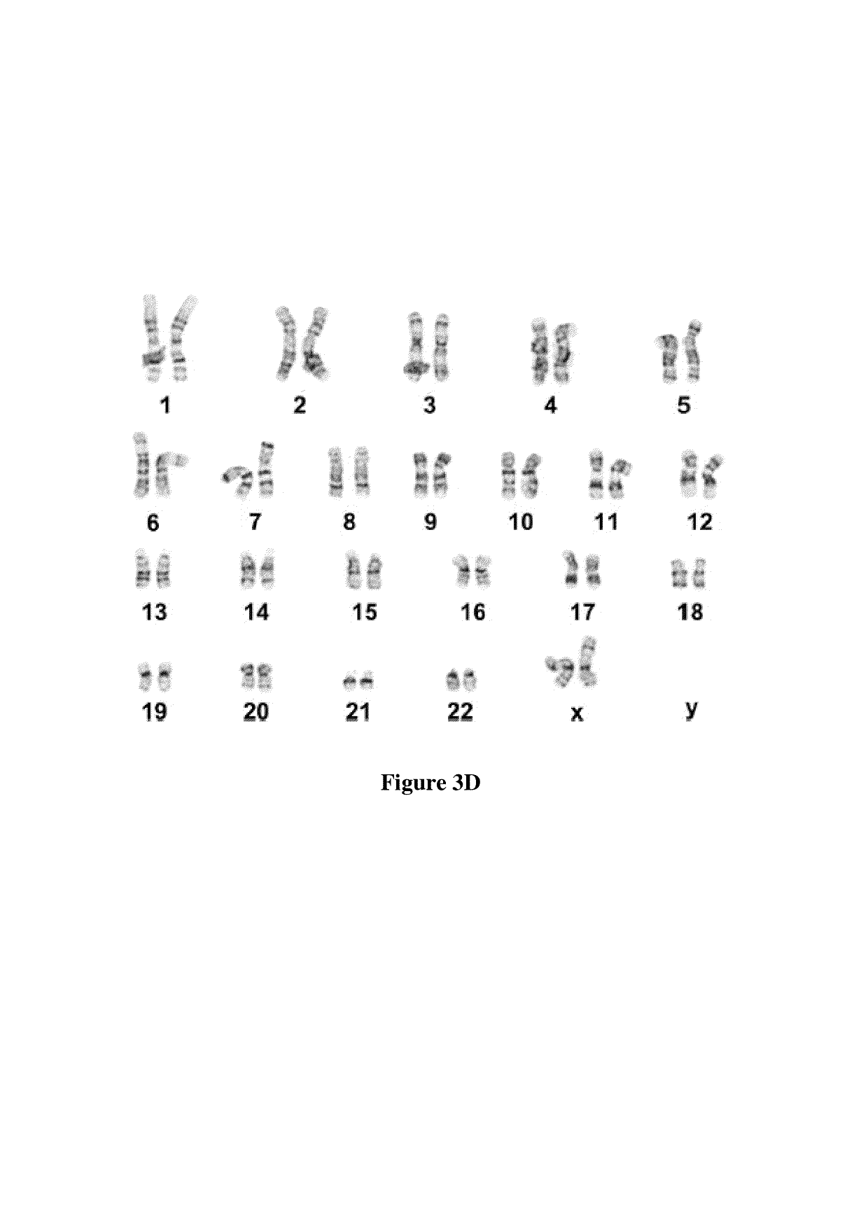

One of the unique characteristics of pluripotent stem cells is their ability to form embryoid bodies (EBs) in suspension culture and differentiate into all the three germ layers. The HUES9 cells grown on PAm₆-co-PSS₂ hydrogels formed EBs (FIG. 3A). The cells expanded on PAm₆-co-PSS₂ matrices were also differentiated into mesoderm, ectoderm, and endoderm, further confirming that the cells grown extensively on these hydrogels maintained their ability to differentiate into multiple germ layers (FIGS. 3B and 3C). Additionally, the cells cultured on PAm₆-co-PSS₂ maintained a normal karyotype (FIG. 3D). Together these findings demonstrate the potential of PAm₆-co-PSS₂ to support long-term culture of undifferentiated hPSCs while maintaining their pluripotency.

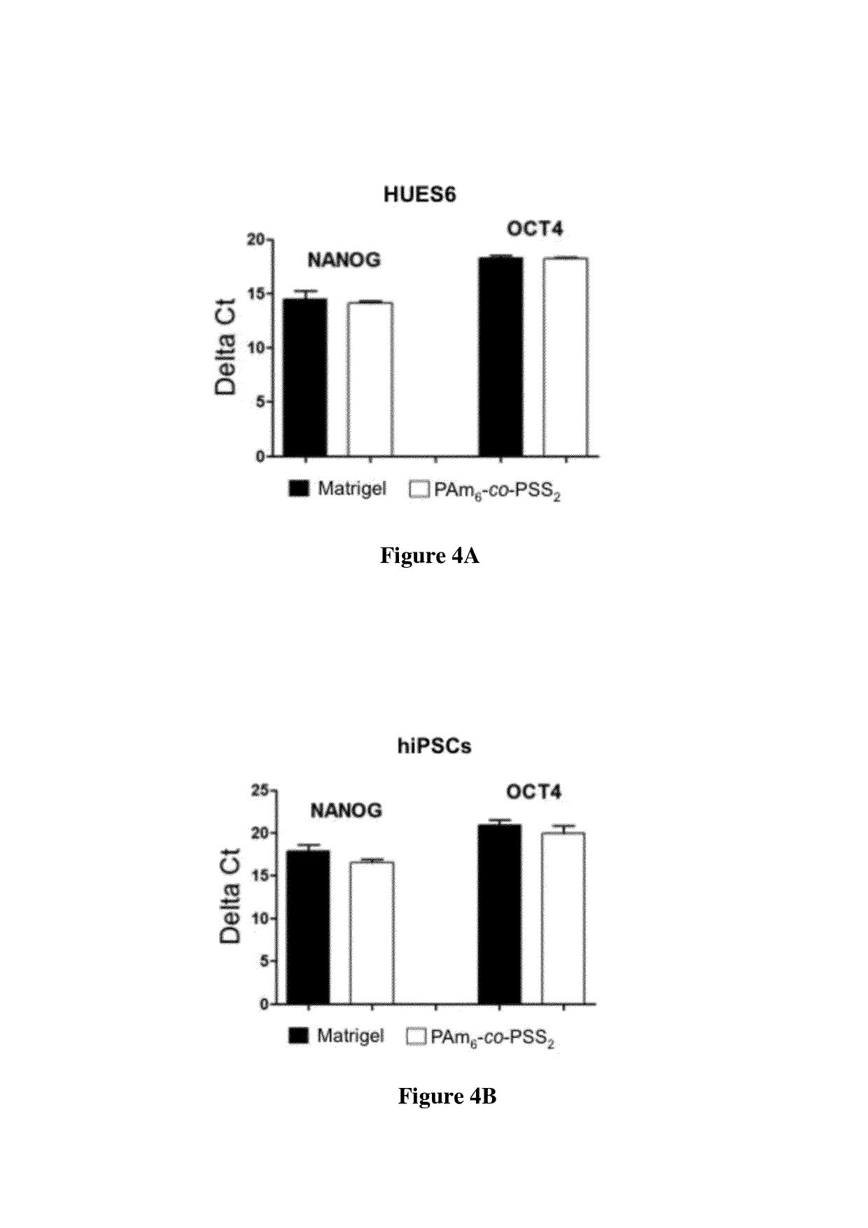

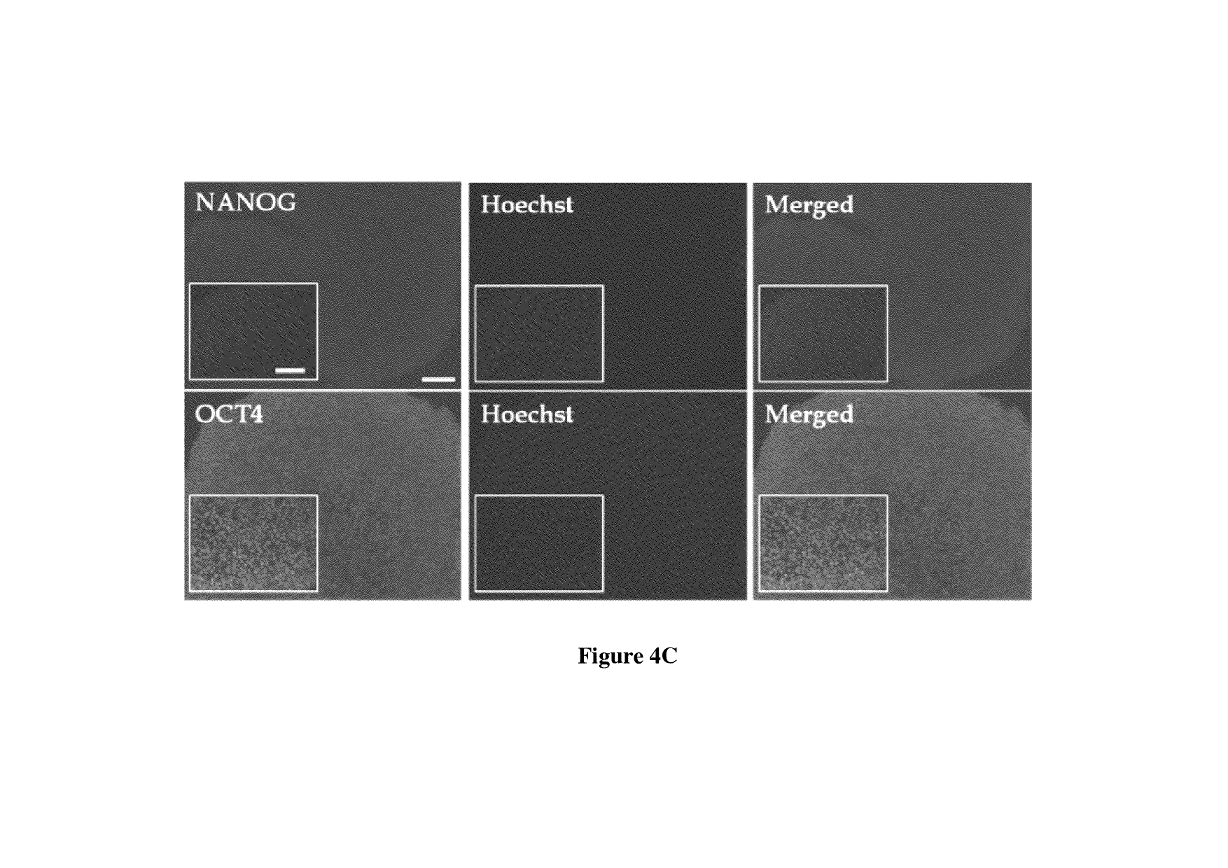

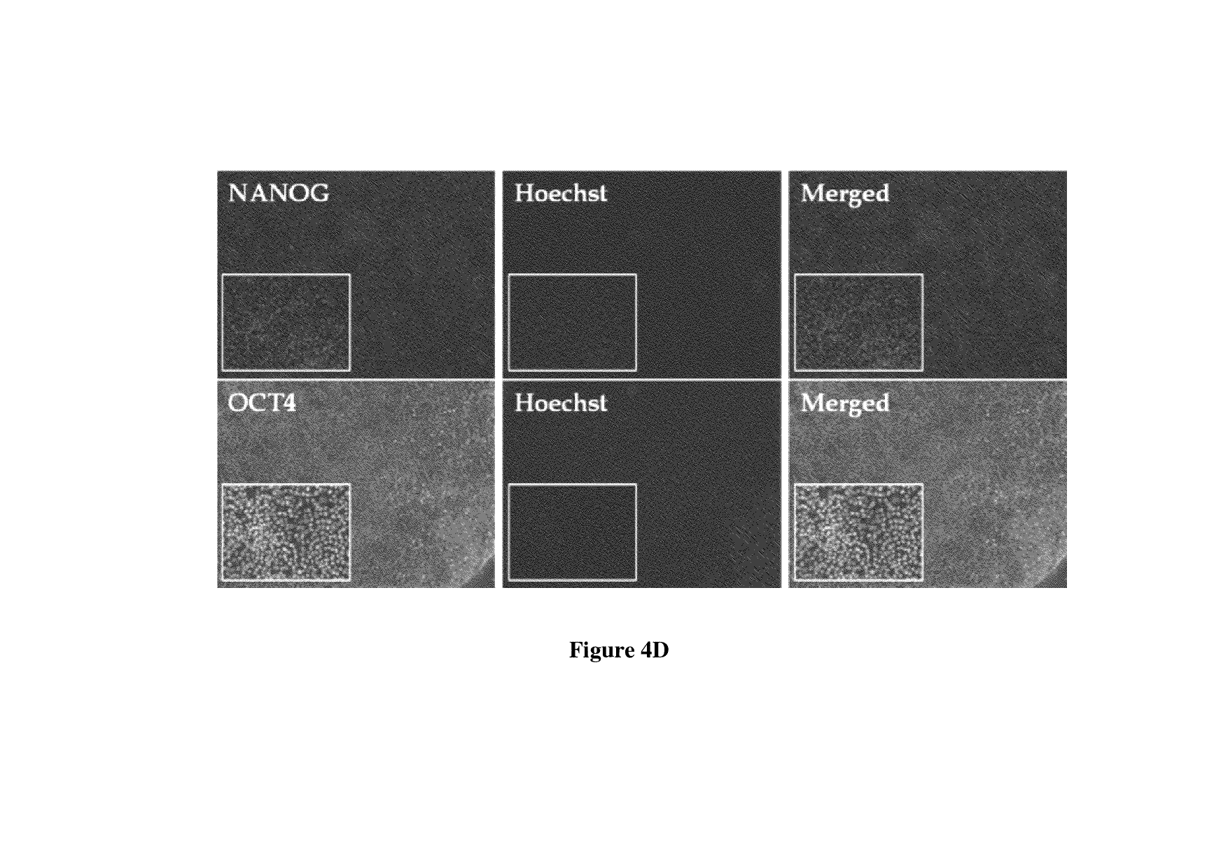

To determine whether the PAm₆-co-PSS₂-assisted self-renewal of HUES9 cells is applicable to other hPSCs, the potential of PAm₆-co-PSS₂ to support the growth of HUES6 and human induced pluripotent stem cells (hiPSCs) was investigated in vitro. Similar to HUES9 cells, HUES6 and hiPSCs cultured and passaged over multiple times on PAm₆-co-PSS₂ hydrogels in StemPro medium displayed characteristic hPSC morphology, bright colony formation, and OCT4 and NANOG expression comparable to Matrigel (FIGS. 4A-4D).

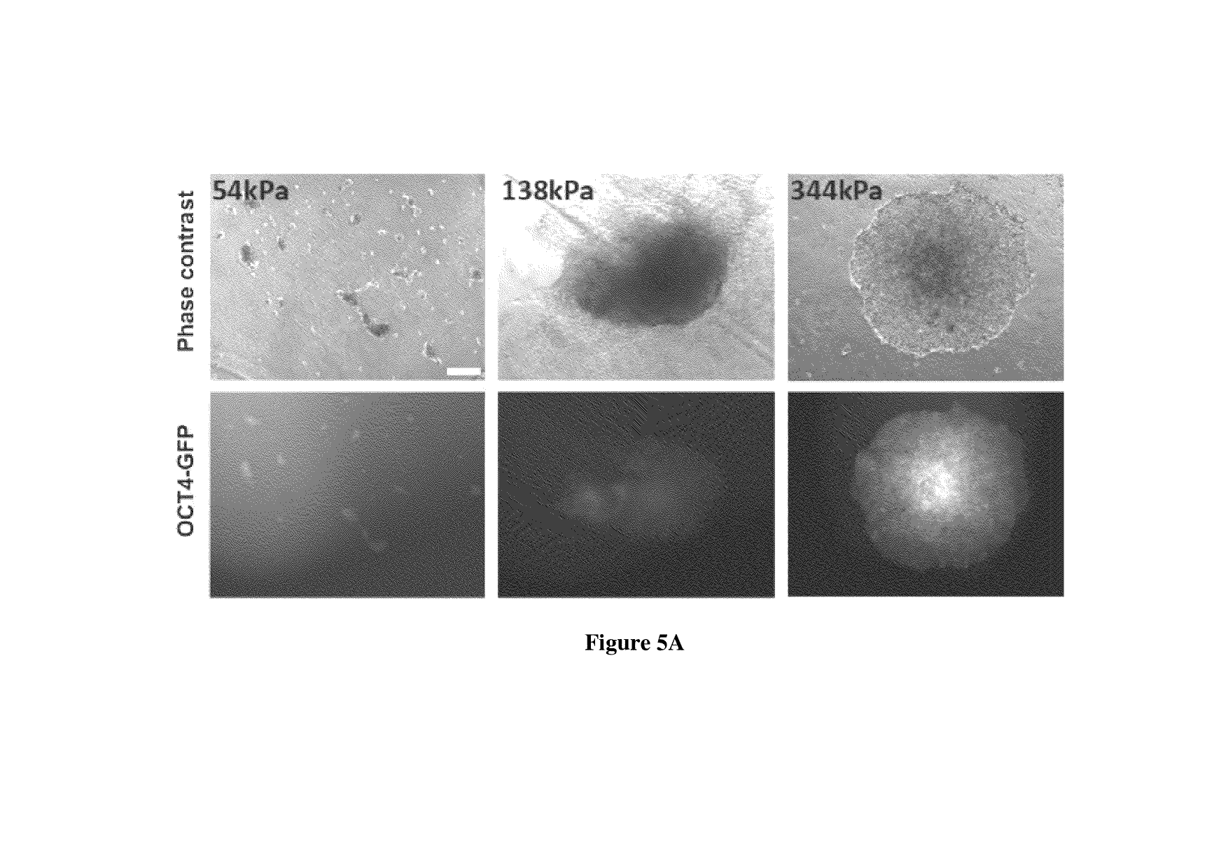

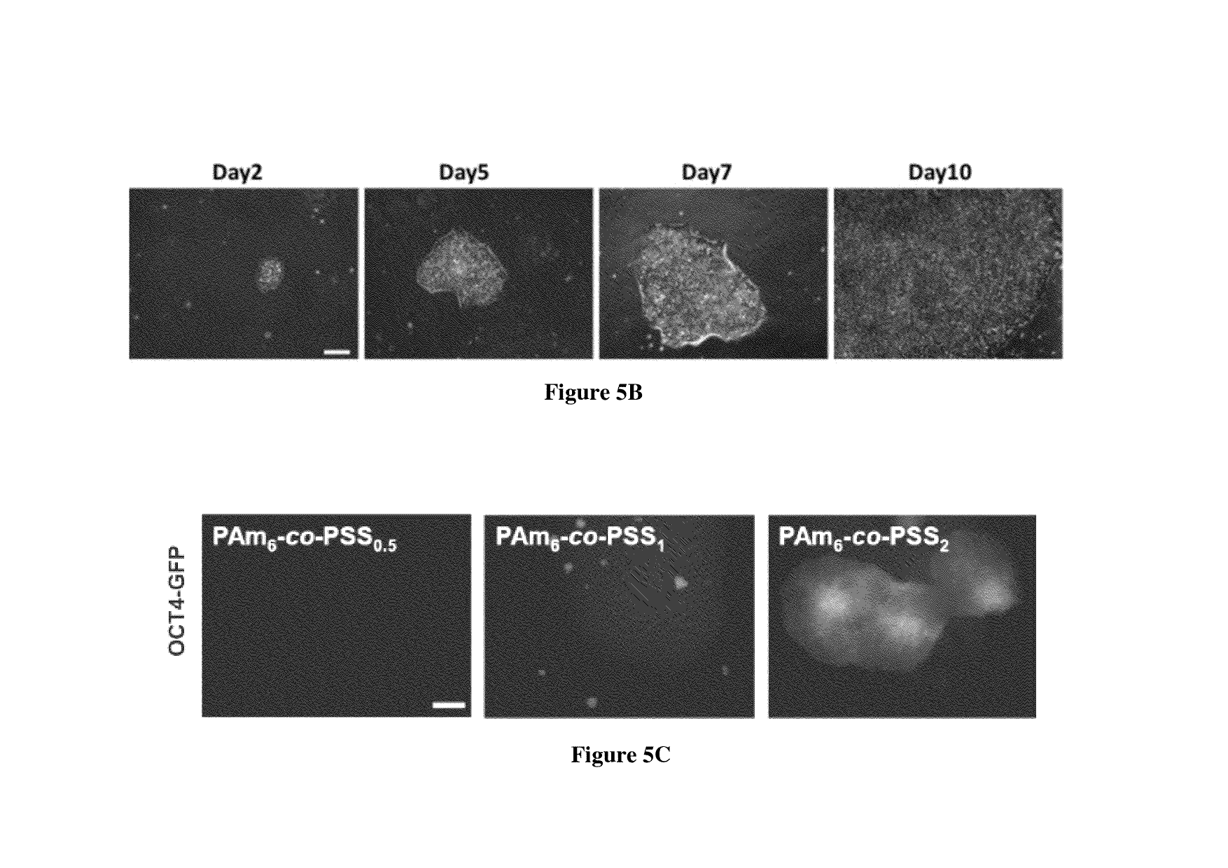

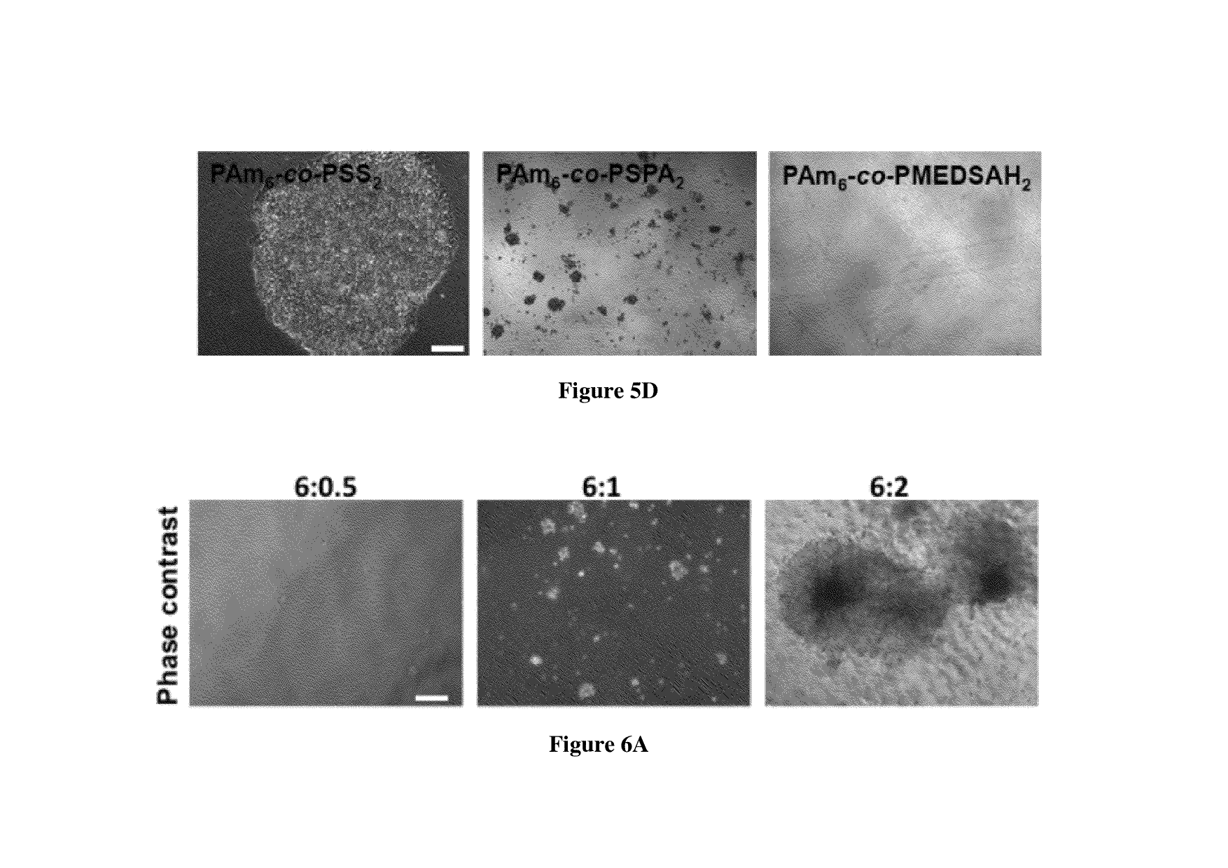

Matrix rigidity on hPSCs—Having established the unique ability of PSS-based hydrogels (PAm₆-co-PSS₂) to support the growth of hPSCs in vitro while maintaining their pluripotency, the effect of matrix rigidity on hPSCs was then determined. Matrix rigidity has been identified as an important material parameter on stem cell fate determination. To examine the role that the hydrogel rigidity plays in hESC attachment and self-renewal, a number of PAm₆-co-PSS₂ hydrogels were synthesized with different bulk moduli (i.e., about 54, about 138, and about 344 kPa), by varying their cross-link density (see Table 1). Note that the changes in modulus also introduced subtle changes in matrix hyrophilicity as rigidity affects swelling, which in turn affects the surface density of sulfonate functional groups accessible at the interface. As seen from FIG. 5A, an increased cell adhesion and colony formation was observed with increasing rigidity of PSS hydrogels. FIG. 5B demonstrates the growth of HUES9 cells on these PAm₆-co-PSS₂ hydrogels having higher elastic modulus (about 344 kPa). PAm₆-co-PSS₂ hydrogels with low bulk rigidity (about 54 kPa) supported minimal cell adhesion while those having a rigidity of about 138 kPa exhibited moderate cell adhesion, but the attached cells underwent spontaneous differentiation.

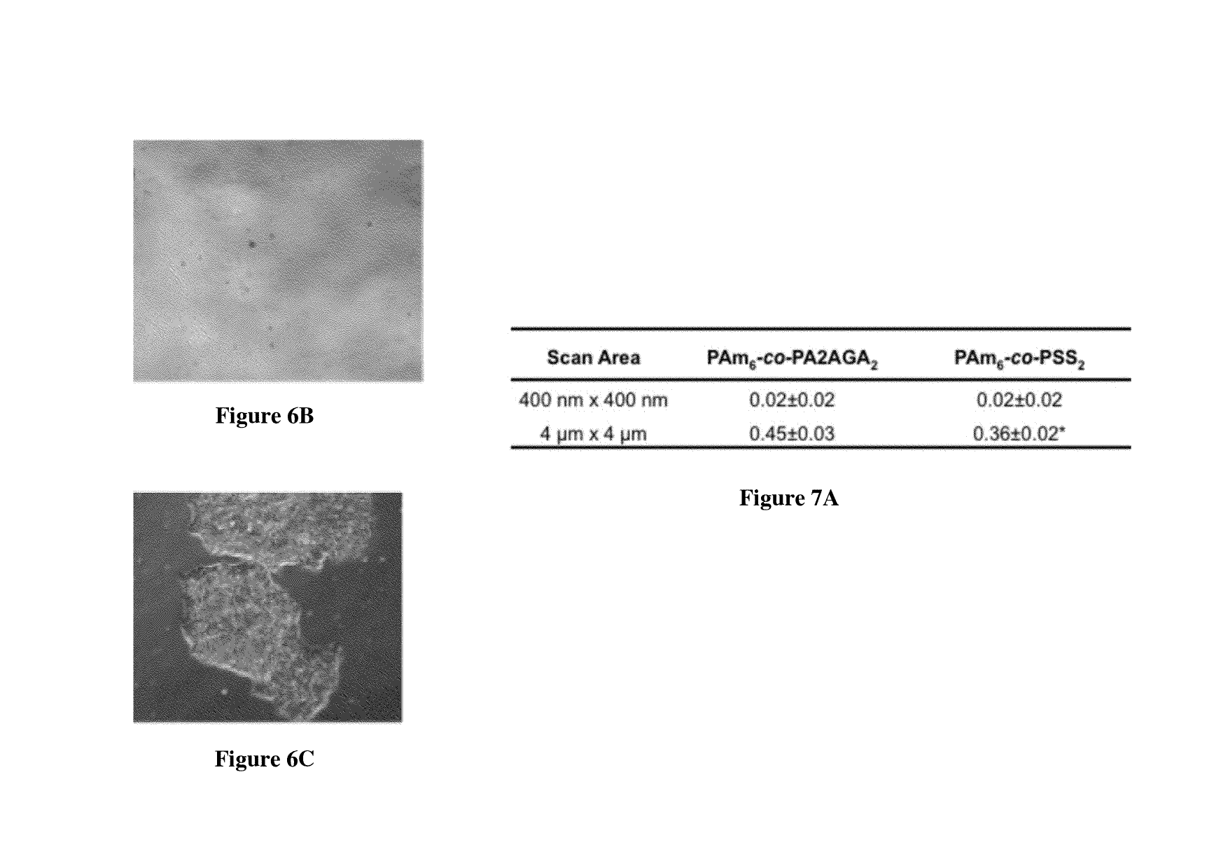

Effect of chemical functional group(s) and matrix hydrophobicity on hPSCs—The effect of hydrogel composition on adhesion and growth of hPSCs was investigated by varying the amount of PSS content within the hydrogels (Am:PSS 6:0.5, 6:1, 6:2). Significant differences in adhesion and colony formation of HUES9-Oct4-GFP cells were observed amongst the hydrogels; specifically a monotonic dependence with the PSS content was observed (FIGS. 5C and 6A). No cell adhesion was observed on hydrogels containing lower amounts of PSS moieties (PAm₆-co-PSS_{0.5}). While an increase in PSS content in the hydrogel (PAm₆-co-PSS₁) supported cell adhesion, they failed to support the colony formation of adhered cells. A further increase in PSS content, as in PAm₆-co-PSS₂, supported both adhesion and colony formation of HUES9-Oct4-GFP cells (FIGS. 5C and 6A). Note that varying the hydrogel composition also introduced subtle changes to their hydrophilicity and bulk rigidity (see Table 1).

Given the importance of functional group and matrix hydrophilicity on cell adhesion, the cellular responses of HUES9 cells on different hydrogels with varying hydrophilicity were also evaluated. These hydrogels were created by reacting Am with different monomers (SS, SPA and MEDSAH) terminating with sulfonate functional group at a mole ratio of 6:2 (Am:comonomer). These hydrogels have similar elastic moduli and functional groups but varying matrix hydrophilicities (Table 1). Similar to PAm₆-co-PSS₂ hydrogels, significant cell adhesion was observed initially on PAm₆-co-PSPA₂ hydrogels, while minimal to no cell adhesion was observed on PAm₆-co-PMEDSAH₂ (FIG. 5D). However, unlike PAm₆-co-PSS₂ hydrogels, cells on PAm₆-co-PSPA₂ did not grow to form bright compact colonies (FIG. 5D).

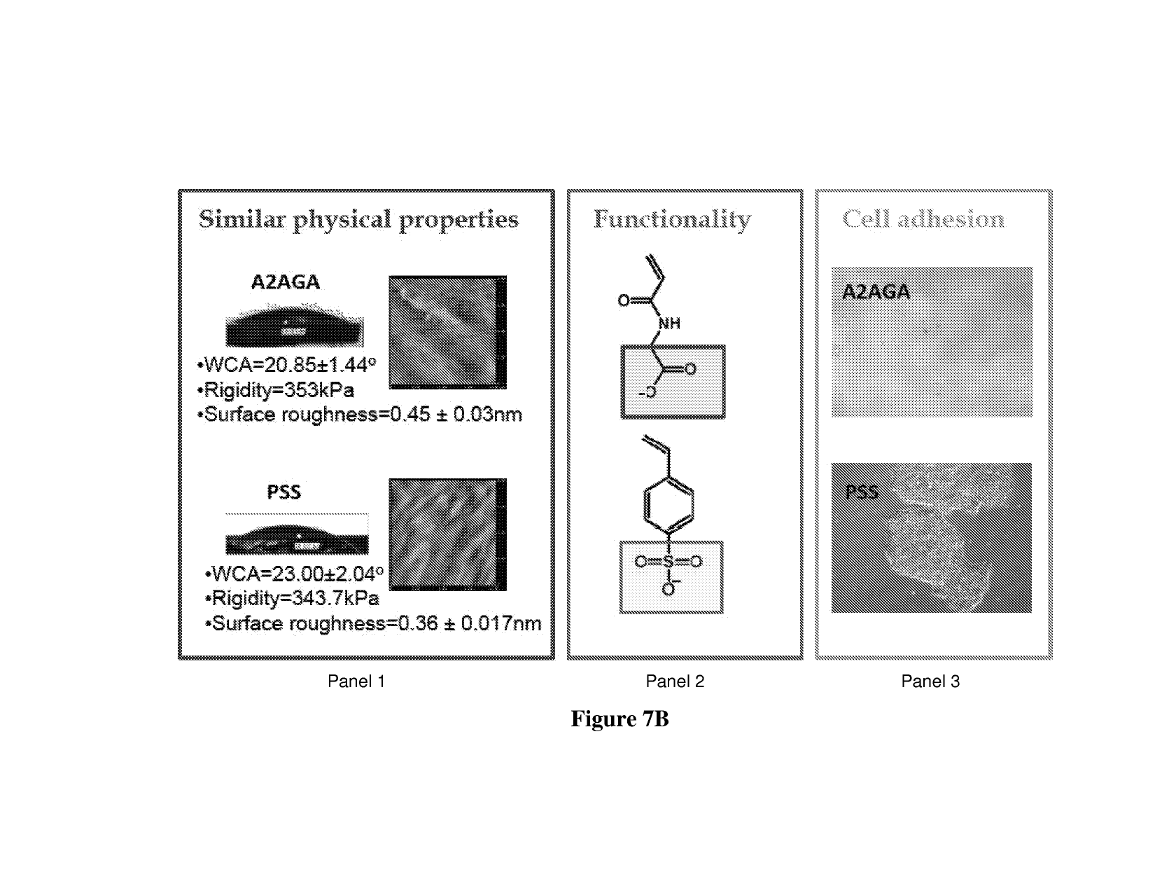

These results clearly indicate the effect of multiple physical and chemical cues of the underlying matrix on hPSC response. In an effort to delineate the effect of various material properties from that of the functional group, copolymer hydrogels bearing carboxylate groups (PAm₆-co-PA2AGA₂) were synthesized having similar elastic modulus, hydrophilicity, and topography to that of PAm₆-co-PSS₂ (Table 1 and FIGS. 7A and 7B). Unlike PAm₆-co-PSS₂, the HUES9 cells on PAm₆-co-PA2AGA₂ hydrogels exhibited minimal to no cell adhesion (FIGS. 6B and 6C). The effect of carboxyl functional groups on hPSCs was also examined by employing different PAm₆-co-PB₂ hydrogels having carboxyl functional groups but varying hydrophilicity (Table 1). Similar to PAm₆-co-PA2AGA₂, no cell adhesion was observed on hydrogels with carboxyl functional groups (data not shown). These findings demonstrate the importance of sulfonate groups on the observed PAm₆-co-PSS₂-mediated cell response.

Cell-matrix interface on adhesion and growth of hPSC—The initial cell-matrix adhesion had a significant effect on endogenous expressions of cell surface integrins and ECM components. These cells were cultured in StemPro and compared against those cultured on Matrigel under identical conditions. HUES9 cells were grown on PAm₆-co-PSS₂ for over 20 passages while HUES6 and hiPSCs were cultured for 15 passages.

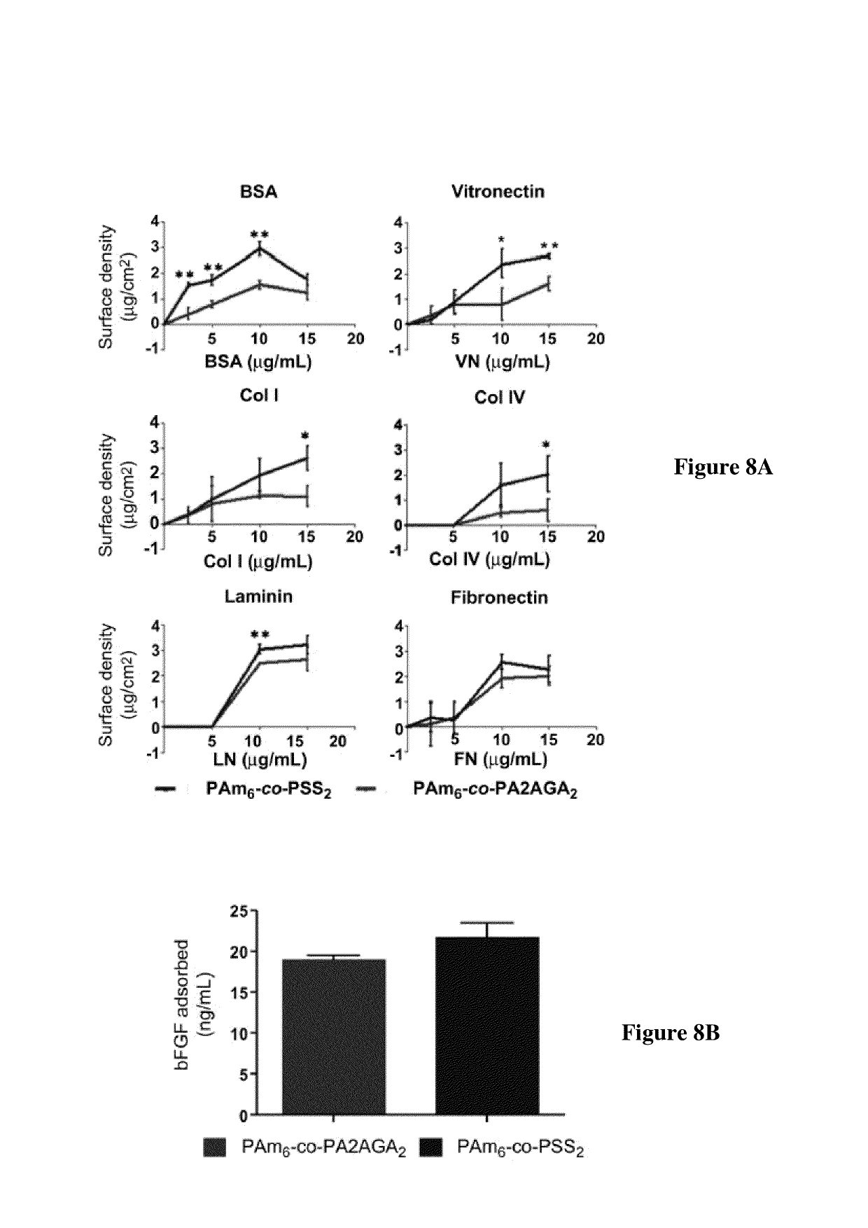

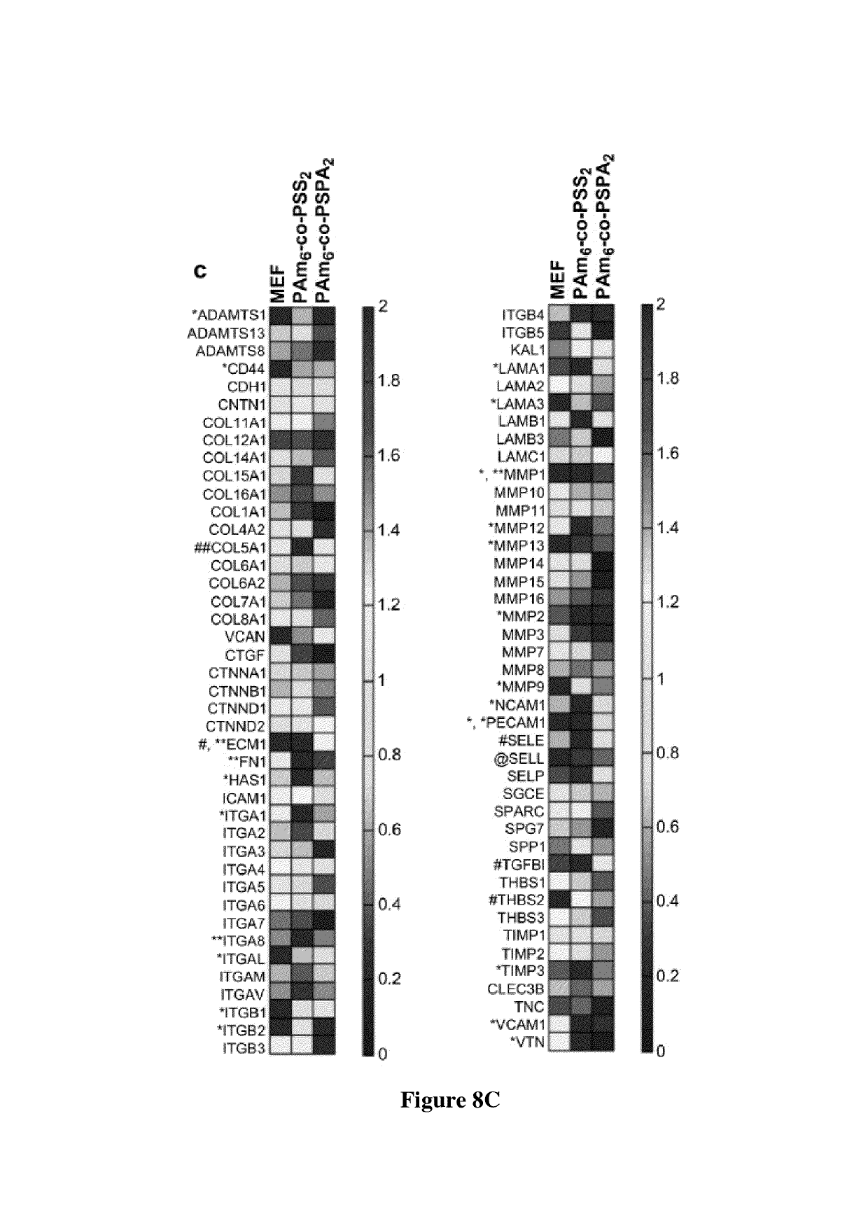

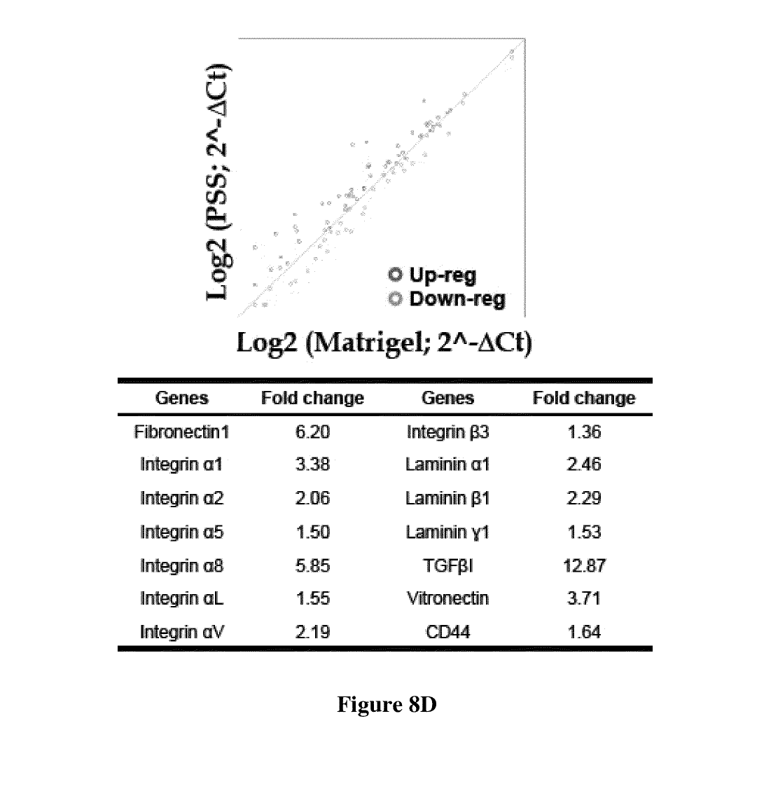

As the interface of the hydrogels was not functionalized with proteins or peptides and a short incubation of the hydrogels in serum medium prior to cell seeding was needed for initial cell adhesion, the adsorption of various extracellular matrix proteins (ECM) onto the hydrogel surfaces was examined. It is well-known the effect of matrix interfacial properties (hydrophilicity, functional group, surface roughness, rigidity, etc.) affect protein adsorption and conformation, thereby influencing cell adhesion. Protein adsorption on PAm₆-co-PSS₂ hydrogels was examined and compared to PAm₆-co-PA2AGA₂. These two hydrogel systems were chosen based on the observation that despite having similar hydrophilicity, surface roughness, and rigidity, PAm₆-co-PSS₂ hydrogels support hPSCs while PAm₆-co-PA2AGA₂ hydrogels do not. The effect of these materials on bFGF binding was also examined. While both of the hydrogels supported adsorption of ECM proteins and bFGF, PAm₆-co-PSS₂ hydrogels consistently exhibited higher protein adsorptions compared to PAm₆-co-PA2AGA₂, especially in the case of certain proteins such as BSA and VN (FIGS. 8A and 8B).

Cell surface adhesion molecules such as integrins play an important role in the initial adhesion of hPSCs to the underlying ECM, and also in the regulation of their self-renewal. Similarly, ECM components secreted by the hPSCs as well as the feeder cells have also been shown to play an important role in maintaining the pluripotency of hPSCs. It was therefore determined that the endogenous expressions levels of various cell surface adhesion molecules and ECM components of HUES9 cells cultured on PAm₆-co-PSS₂, PAm₆-co-PSPA₂, and compared the results against those of MEF- and Matrigel-supported culture under identical conditions. The PAm₆-co-PSPA₂ hydrogel was chosen as it supported initial adhesion of HUES9 cells similar to PAm₆-co-PSS₂, but failed to support their growth and colony formation. As seen from FIGS. 8C and 8D, the underlying matrix had a significant effect on the gene expression profile of the cells. In short, the cells on PAm₆-co-PSS₂ hydrogels exhibited higher expression levels of various integrins and ECM proteins that are known to be relevant to self-renewal of hPSCs. Specifically, HUES9 cells cultured on PAm₆-co-PSS₂ expressed higher levels of fibronectin, laminin, collagen, and vitronectin, as well as integrins α₁, α₂, α₈, and αᵥ. Many of these ECM proteins and integrins have been implicated to play an important role in self-renewal of hPSCs. Additionally, cells on PAm₆-co-PSS₂ hydrogels expressed higher levels of MMP family of proteins indicating the potential role of ECM remodeling.

Together, the results provided herein demonstrate that the adhesive interface of the PAm₆-co-PSS₂ matrices, mediated through protein adsorption, supports initial adhesion of hPSCs, which in turn facilitates both cell-matrix and cell-cell interactions to allow colony formation of the adhered cells. While the adsorbed proteins support initial adhesion of seeded cells, without being bound by theory, it is likely that the cell-secreted ECM proteins are the ones that support long-term maintenance and growth of these cells, as shown by the transcription profile. The reciprocal interactions of cells with their surrounding ECM play an important role in their fate determination as ECM components can induce various intracellular signals to drive self-renewal vs. differentiation decisions. Recent studies have indicated the importance of a combination of integrins and ECM proteins in maintaining stemness of pluripotent cells. For instance, a recent study demonstrated the superior effect of matrices comprising of several peptides over that of single peptide on supporting self-renewal of hESCs. A similar finding was also reported which showed the beneficial effect of a combination of ECM proteins on supporting self-renewal of hESCs. In addition to ECM proteins and integrins, the cells cultured on PAm₆-co-PSS₂ hydrogels exhibit higher levels of MMP proteins indicating potential ECM remodeling in hPSC self-renewal. This result is consistent with a recent study, which demonstrated the role of ECM remodeling and endogenous cell-secreted factors on self-renewal of mouse embryonic stem cells (mESCs). Any perturbations to the ESC-secreted signaling resulted in the mESCs exiting their self-renewal state, thus demonstrating the importance of autocrine factors on self-renewal of pluripotent stem cells.

As such, the findings provided herein demonstrate that synthetic hydrogels having a combination of physicochemical properties support adhesion and growth of hPSCs by activating cellular processes and harnessing autocrine factors that are conducive for self-renewal of hPSCs. The hydrogel-based synthetic matrices introduced here support adhesion of hPSCs and their long-term growth without compromising their pluripotency and karyotypic stability. Such tunable synthetic matrices also serve as platforms to elucidate the roles of different biophysical and biochemical cues in cell-matrix and cell-cell interactions.

The following examples are intended to illustrate but not limit the invention.

### EXAMPLE 1

### Materials and Methods

Materials: The following monomers were used. N-acryloyl amino acid (AA) monomers, such as N-acryloyl 2-glycine (A2AGA), N-acryloyl 4-aminobutyeric acid (A4ABA), N-acryloyl 6-aminocaproic acid (A6ACA), and N-acryloyl 8-aminocaprylic acid (A8ACA), were synthesized from glycine (Fisher Scientific, Inc.), 4-aminobutyeric acid, 6-aminocaproic acid, and 8-aminocaprylic acid (Acros Organics Inc.). Sodium 4-vinylbenzenesulfonate (SS), 3-sulfopropyl acrylate potassium salt (SPA), and [2-(methacryloyloxy)ethyl]dimethyl-(3-sulfopropyl)ammonium hydroxide (MEDSAH) were purchased from Aldrich. Acrylamide (Am) was purchased from Invitrogen and N,N′-methylenebisacrylamide (BisAm), ammonium persulfate (APS) and N,N,N′,N′-tetramethylethylenediamine (TEMED) were obtained from Sigma.

Hydrogel preparation: The hydrogels containing varying functional groups and hydrophilicity were synthesized through copolymerization of acrylamide with monomers containing either carboxylate or sulfonate groups. The PSS-based hydrogels (PAm₆-co-PSS₂, PAm6-co-PSS₁, PAm₆-co-PSS_{0.5}) were synthesized by copolymerizing acrylamide (Am, 7.5 mmol) with sodium 4-vinylbenzenesulfonate (SS, 2.5 mmol) monomers at 6:2, 6:1, and 6:0.5 mole ratios. The monomers were dissolved in deionized (DI) water, and polymerized in BioRad 1 mm spacer glass plates at room temperature using 0.26, 0.19, and 0.10 mmol BisAm as a crosslinker and 1.3% w/v APS/TEMED (redox initiator/accelerator). Hydrogels containing SPA and MEDSAH moieties (PAm₆-co-PSPA₂, PAm₆-co-PMEDSAH₂ were synthesized by copolymerizing Am (7.5 mmol) with SPA (2.5 mmol) or MEDSAH (2.5 mmol) at a mole ratio of 6:2. The precursors were dissolved in DI water and polymerized using 0.26 mmol BisAm and 1.3% w/v APS/TEMED. Lastly, hydrogels with carboxyl groups were synthesized by copolymerizing Am (7.5 mmol) with AA monomers (2.5 mmol) at a mole ratio of 6:2. The monomers were dissolved in 1M NaOH and polymerized using 0.26 mmol BisAm and 1.3% w/v APS/TEMED. The compositions and nomenclature of the hydrogels are summarized in Table 1. The hydrogels were sterilized with 70% ethanol and washed with fresh phosphate buffered saline (PBS) solution for 72 hours. The rinsed hydrogels were incubated in culture media (high glucose DMEM with 2 mM L-glutamine and 50 units/ml penicillin/streptomycin) containing 10% fetal bovine serum (premium select) overnight before plating cells.

TABLE 1
Composition, Nomenclature, and Characterization of the Hydrogels

| | | Mole ratio | Elastic | |
|---|---|---|---|---|
| | Comonomer | of Amₓ:B_{y} | modulus | Water contact |
| Sample code | (B) | (BisAm) | (kPa) | angle (°) |
| PAm₆-co-PA2AGA₂ | A2AGA | 6:2 (0.26) | 353.0 ± 13.9 | 20.9 ± 1.4 |
| | (2.5 mmol) | | | |
| PAm₆-co-PA4ABA₂ | A4ABA | 6:2 (0.26) | N/A | 36.1 ± 1.9 |
| | (2.5 mmol) | | | |
| PAm₆-co-PA6ACA₂ | A6ACA | 6:2 (0.26) | N/A | 53.8 ± 2.7 |
| | (2.5 mmol) | | | |
| PAm₆-co-PA8ACA₂ | A8ACA | 6:2 (0.26) | N/A | 67.3 ± 1.6 |
| | (2.5 mmol) | | | |
| PAm₆-co-PSPA₂ | SPA (2.5 mmol) | 6:2 (0.26) | 342.5 ± 19.7 | 32.6 ± 1.2 |
| PAm₆-co-PMEDSAH₂ | MEDSAH | 6:2 (0.26) | 378.8 ± 36.6 | 66.3 ± 2.3 |
| | (2.5 mmol) | | | |
| PAm₆-co-PSS_{0.5} | SS | 6:0.5 (0.26) | 327.8 ± 7.1 | 14.3 ± 1.7 |
| | (0.625 mmol) | | | |
| PAm₆-co-PSS₁ | SS (1.25 mmol) | 6:1 (0.26) | 382.9 ± 5.1 | 17.4 ± 0.3 |
| PAm₆-co-PSS₂ | SS (2.5 mmol) | 6:2 (0.26) | 343.7 ± 5.1 | 23.0 ± 2.0 |
| PAm₆-co-PSS₂-M* | SS (2.5 mmol) | 6:2 (0.19) | 137.6 ± 6.9 | 20.8 ± 0.5 |
| PAm₆-co-PSS₂-L* | SS (2.5 mmol) | 6:2 (0.10) | 53.5 ± 2.8 | 17.5 ± 1.5 |

*PAm₆-co-PSS₂-M and PAm₆-co-PSS₂-L represent PAm₆-co-PSS₂ hydrogels with an elastic modulus of 137.6 ± 6.9 kPa and 53.5 ± 2.8 kPa, respectively.

Surface roughness: Surface roughness of the hydrogels was evaluated using a Multimode AFM equipped with a Nanoscope IIIA controller from Veeco Instruments (Santa Barbara, Calif.) run by Nanoscope software v5.30. AFM images were acquired in contact mode at forces of ˜4 nN with an “E” scanner (maximum scan area 12×12 mm²) using Si₃N₄ cantilevers (Veeco) with 0.06 N/m nominal spring constants. Hydrogels were prepared as described above. Hydrogels were prepared as described above. Upon synthesis, the hydrogels were washed in PBS for 36 hours to leach out the unreacted monomers and to reach equilibrium swelling. For a given scan area, the reported roughness value is the average root mean square (RMS) roughness obtained from two different spots of triplicate specimens. Using the nanoscope software, data analysis was carried out where a flattening order 3 was applied to all images to correct for tilt and bow before roughness analysis.

Elastic modulus: Equilibrium swollen hydrogels were used for compression measurements. The measurements were performed using Bose ElectroForce 3200 Test Instrument (Bose, Minn., USA). Samples were compressed by two parallel plates at the maximum loading of 225N load cell with a crosshead speed of 0.1 mm/min. The elastic moduli were calculated from the linear region of stress-strain curve (0-5% strain). All measurements were carried out as quadruplicates for each set of parameters.

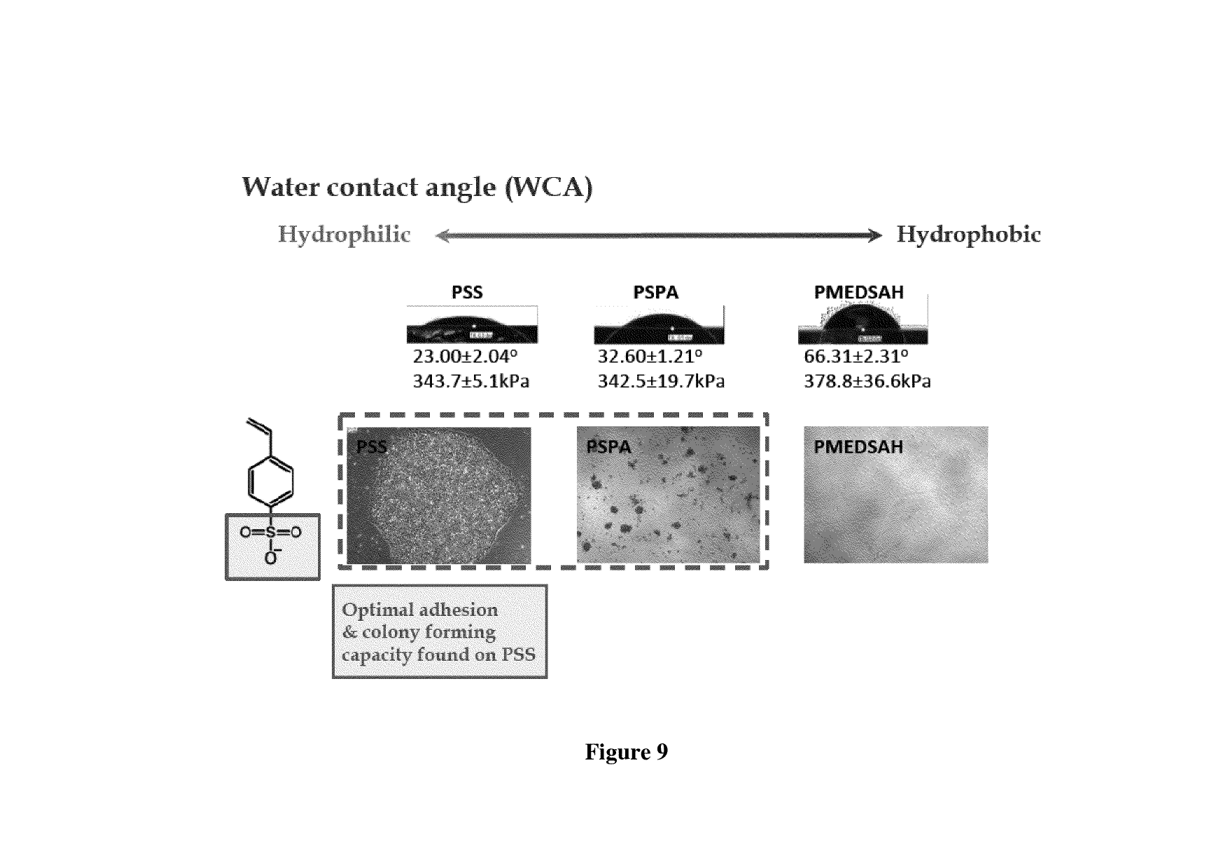

Water contact angle: The water contact angle of the hydrogels was determined by a sessile drop method at room temperature using a contact angle meter (CAM100, KSV Instruments Ltd.). A 5 μL droplet of water was placed on the surface of hydrogels (FIG. 9). All samples were prepared as triplicates and results were shown as a mean value with standard deviation.

HUES9-Oct4-GFP: A lentiviral construct was obtained to generate the Oct4-GFP reporter line. The reporter line was generated as described earlier. The HUES9 cells were infected overnight with lenti Oct4-GFP and single clones were isolated and screened for (i) stable GFP expression levels, (ii) low GFP expression levels after EB formation, and (iii) rapid decrease in GFP expression upon removal of MEF conditioned medium.

Culture of HPSCs: HUES9, HUES9-Oct4-GFP, HUES6, and human induced pluripotent stem cells (hiPSCs), were expanded in defined medium (StemPro®; DMEM/F-12 supplemented with StemPro supplement, 2% bovine serum albumin (BSA), 55 μM 2-mercaptoethanol, and 1% Gluta-MAX) or in MEF conditioned medium. The MEF conditioned medium was collected after culturing MEF for 24 hours using growth medium (Knockout DMEM supplemented with 10% Knockout Serum Replacement, 10% human plasmonate (Talecris Biotherapeutics), 1% non-essential amino acids, 1% penicillin/streptomycin, 1% Gluta-MAX, 55 μM 2-mercaptoethanol as described elsewhere. The hPSCs were manually passaged as small clumps of 30-40 μm size after 6 days of culture onto different matrices (Matrigel and synthetic matrices) by using a splitting ratio of 1:4. All the sequential passages were carried out similarly by passaging the cells manually. The hPSCs on PAm₆-co-PSS₂ hydrogels were passaged after 10-12 days depending upon the colony size and morphology. All cultures were supplemented with fresh medium containing 30 ng/ml of bFHF (Life Technologies) daily.

Population doubling time (PDT): Population doubling time (PDT) of HUES9 cells grown on Matrigel, MEF, and PAm₆-co-PSS₂ hydrogel was calculated using the equation below:

$$P D T ( hr ) = \frac{( T 2 - T 1 )}{3.32 * ( log N 2 - log N 1 )}$$

where T1 was day 3, T2 was day 5, N1 was the number of cells at T1, and N2 was the number of cells at T2. The number of cells at each time point was counted using TC10™ Automated Cell Counter (Biorad).

For PDT measurements, HUES9 cells were cultured as single cells by enzymatically splitting the cells using Accutase. The cell count was carried out after 3 and 5 days of culture to calculate the population doubling time.

Immunocytochemistry: Immunofluorescent staining was performed using the following primary antibodies: OCT4 (1:200; Santa Cruz), NANOG (1:200; Santa Cruz), SOX17 (1:200; R & D systems), SMA (1:500; R & D systems), and NESTIN (1:50; BD Biosciences). The following secondary antibodies were used: goat anti-rabbit Alexa 647 (1:400; Life Technologies), donkey anti-mouse Alexa 546 (1:250; Life Technologies), and donkey anti-goat Alexa 546 (1:250; Life Technologies). For immunofluorescent staining, cells were fixed in 4% PFA for 5 min at 4° C., followed by 10 min at room temperature. Immediately before staining, the cells were permeabilized with 0.2% (v/v) Triton X-100 and blocked with 1% (w/v) BSA and 3% (w/v) nonfat dry milk for 30 min. Cells were stained with the primary antibodies diluted in 1% BSA overnight at 4° C., washed 3 times with TBS, and incubated with secondary antibodies for 1 hr at 37° C. The nuclei were stained with Hoechst 33342 (2 μg/ml; Life Technologies) for 5 min at room temperature. Imaging was performed using an automated confocal microscope (Olympus Fluoview 1000 with motorized stage and incubation chamber).

RNA isolation and quantitative PCR: RNA isolation was carried out by using TRIzol (Invitrogen), and treated with DNase I (Invitrogen). Reverse transcription was performed by means of qScript cDNA Supermix (Quanta Biosciences). Quantitative PCR was carried out using TaqMan probes (Applied Biosystems) and TaqMan Fast Universal PCR Master Mix (Applied Biosystems) on a 7900HT Real Time PCR machine (Applied Biosystems). Taqman gene expression assay primers (Applied Biosystems) listed in Table 2 were used. Gene expression was normalized to 18S rRNA levels. Delta Ct values were calculated as Cₜ^{target}−Cₜ^{18s}. All experiments were performed with three biological replicates. The relative fold changes in expression were calculated as 2^{-ΔΔCt}. Data are presented as the average of the biological replicates±standard error of the mean.

TABLE 2
List of Primers Used for qRT-PCR

| Gene Symbol | Assay ID |
|---|---|
| SOX17 | Hs00751752_s1 |
| FOXA2 | Hs0023764_m1 |
| CXCR4 | Hs00607978_s1 |
| SOX2 | Hs01053049_s1 |
| Nestin | Hs00707120_s1 |
| SOX1 | Hs01057642_s1 |
| SMA | Hs00426835_g1 |
| ACTC | Hs01109515_m1 |
| NANOG | Hs04260366_g1 |
| OCT4 | Hs00999634_gH |

FACS analysis: hPSCs were dissociated with Accutase. The cells were re-suspended in buffer (2% FBS/0.09% sodium azide/DPBS; BD Biosciences) and stained directly with Alexa-647 conjugated Tra-1-81 (Biolegend) or Alexa Fluor 647 mouse IgM,κ isotype control. Cells were stained for 30 min on ice, washed, and re-suspended in buffer. Samples were analyzed using BD Biosystems FACSCanto.

Embryoid Body Formation: The hPSCs cultured on PAm₆-co-PSS₂ were Accutased for 2-3 min and re-suspended in growth medium without the supplementation of bFGF, plated onto ultra-low attachment plates, and cultured in a 37° C./5% CO₂ incubator for 8 days to form embryoid bodies (EBs).

In vitro differentiation: All media components were procured from Life Technologies unless indicated otherwise. For endoderm differentiation, hPSCs were cultured on Matrigel in mouse embryonic fibroblast (MEF)-conditioned media (CM) supplemented with 30 ng/ml FGF2 until confluency. The medium was then changed to RPMI medium supplemented with 1% (v/v) Gluta-MAX and 100 ng/ml recombinant human Activin A (R&D Systems). Cells were cultured for 3 days, with FBS concentrations at 0% for the first day and 0.2% for the second and third days. Cultures were supplemented with 30 ng/ml purified mouse Wnt3a on the first day.

To initiate ectoderm differentiation, hPSCs were cultured on Matrigel in MEF conditioned medium supplemented with 30 ng/ml FGF2. Cells were then detached by treatment with Accutase (Millipore) for 5 min and re-suspended in neural progenitor cell (NPC) embryoid body (EB) media (10% FBS, 1% N2, 1% B27, DMEM/F-12), 5 μM ROCK inhibitor (Y-27632, Stemgent), 50 ng/ml recombinant mouse Noggin (R&D Systems), 0.5 μM dorsomorphin (Tocris Bioscience). Approximately 7.5×10⁵ cells suspended in neutral progenitor cell medium were added to each well of several 6-well ultra-low attachment plates (Corning). The plates were then placed on an orbital shaker at 95 rpm in a 37° C./5% CO₂ incubator overnight. The formed spherical clusters were then cultured in neural progenitor cell medium supplemented with 50 ng/ml recombinant mouse Noggin and 0.5 μM Dorsomorphin, but no FBS. The medium was subsequently changed every other day. After 5 days in suspension culture, the EBs were transferred to a 10 cm dish (3×6 wells per 10 cm dish) coated with growth factor-reduced Matrigel (1:25 in KnockOut DMEM; BD Biosciences) for attachment. The plated EBs were cultured in NPC EB medium supplemented with 50 ng/ml recombinant mouse Noggin and 0.5 μM Dorsomorphin. After 7 days of attachment, rosette-forming EBs were collected by manual dissection. Isolated rosettes were incubated in Accutase for 15 minutes in a 37° C./5% CO₂ tissue culture incubator. The rosettes were then plated to poly-L-ornithine (PLO; 10 μg/ml; Sigma) and mouse laminin (Ln; 5 μg/ml) coated plates in NPC expansion medium (1% N2, 1% B27, DMEM/F-12) supplemented with 30 ng/ml FGF2 and 30 ng/ml EGF (R & D systems). NPCs were maintained on PLO/Ln plates in NPC media supplemented with 30 ng/ml FGF2 and 30 ng/ml EGF. For mesoderm induction, hPSCs were cultured on Matrigel in DMEM supplemented with 20% FBS and 1% penicillin/streptomycin for 21 days.

Karyotype analysis: To monitor genomic integrity, cells grown on PAm₆-co-PSS₂ hydrogel with StemPro® medium or MEF-conditioned medium were evaluated by cytogenetic analysis at passages 16 and 20 using standard protocols for G-banding (Cell Line Genetics).

### EXAMPLE 2

### PCR Array Analysis for Various Extracellular Matrix Proteins

Briefly, RNA was isolated from cells using TRIzol (Invitrogen), and treated with DNase I (Invitrogen) to remove traces of DNA. Reverse transcription was performed by using RT² First Strand Kit (SABioscience, Cat #330401) and 200 ng of cDNA was processed for quantitative real-time RT-PCR of 84 genes involved in extracellular matrix and adhesion molecules by using PCR array kit (RT² Profiler™ PCR Arrays Extracellular matrix and adhesion molecules, PAHS-013A-2, SABioscience) an ABI Prism 7700 Sequence Detection System (Applied Biosystems). PCR products were quantified by measuring SYBR Green fluorescent dye incorporation with ROX dye reference.

Protein adsorption: The amount of various protein adsorptions to PAm₆-co-A2AGA₂ and PAm₆-co-PSS₂ hydrogels was quantified by a modified Bradford protein assay using Bio-Rad Protein Assay kit (Cat #500-0006). Briefly, the dye agent was prepared according to the manufacturer's protocol, and both hydrogels were synthesized and placed in 96-well plate (n=3). These hydrogels were incubated with 200 μl of bovine serum albumin (Sigma, Cat #A8412), vitronectin (Sigma, Cat #V8379-50UG), collagen type I (BD Biosciences, Cat #354231), collagen type IV (Sigma, Cat #C5533), laminin (Sigma, L6274), and fibronectin (Gibco, Cat #33016-015) solutions diluted at varying concentrations (0, 2.5, 5, 10, and 15 μg/ml) in PBS for 15 hours at 4° C. 30 μl of each supernatant solution was mixed with 200 μl of Bradford dye reagent solution, which was prepared according to manufacturer's protocol. 100 μl of the above solution was transferred to a flat-bottom 96-well plate to measure their absorbance at 595 nm using a Multimode Detector (Beckman Coulter, DTX 880). Three biological replicates were used for the measurements. The adsorption was calculated from a standard curve generated for each corresponding protein of known concentrations.

Enzyme-Linked Immunosorbent Assay (ELISA): The amount of bFGF absorbed by PAm₆-co-A2AGA₂ and PAm₆-co-PSS ₂ hydrogels was carried out by bFGF ELISA kit (RayBiotech, Inc., cat #ELH-bFGF-001) following the manufacturer's protocol. Similar to the protein adsorption assay, circular hydrogels measuring 6 mm in diameter were prepared and placed onto a 96-well plate. These hydrogels were incubated with 250 ml of bFGF (30 ng/ml) at 37° C. for approximately 12 h 100 ml of the each supernatant solution was transferred to a bFGF microplate (96-wells coated with anti-human bFGF) and incubated overnight at 4° C., followed by incubation with a biotinylated antibody and streptavidin solution. After washing, 100 ml of a TMB substrate solution was added to the wells and samples were incubated for 30 min. Finally, 50 ml of the stop solution was added to samples and their absorbance at 450 nm was measured by using a Multimode Detector (Beckman Coulter, DTX 880). Three biological replicates were used for the measurements. The adsorption was calculated from a standard curve generated by bFGF standards provided by the manufacturer.

Statistical Analysis: All values were presented as mean±standard deviation and statistical significance was determined by two-tailed unpaired Student's t-test.

While the disclosure has been described with reference to the above examples, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth. Accordingly, the invention is limited only by the following claims.

What is claimed is:

1. A synthetic hydrogel comprising a heparin mimetic moiety and an acrylamide monomer.

2. The synthetic hydrogel of claim 1, wherein the heparin mimetic moiety is poly(sodium-4-styrenesulfonate) (PSS).

3. The synthetic hydrogel of claim 2, wherein the molar fraction of PSS ranges from about 0.5 to 2.

4. The synthetic hydrogel of claim 2, wherein the PSS has a matrix rigidity of about 54 kPa, about 138 kPa, or about 344 kPa.

5. The synthetic hydrogel of claim 3, wherein the molar ratio of the acrylamide monomer to PSS is 6:0.5, 6:1, or 6:2.

6. The synthetic hydrogel of claim 5, wherein the molar ratio of the acrylamide monomer to PSS is 6:2.

7. The synthetic hydrogel of claim 1, further comprising a bisacrylamide.

8. A method for synthesizing a hydrogel comprising copolymerizing an acrylamide monomer with an ionic monomer.

9. The method of claim 8, wherein the ionic monomer is sodium-4-styrenesulfonate (SS).

10. The method of claim 8, wherein the ionic monomer is poly(sodium-4-styrenesulfonate) (PSS).

11. The method of claim 10, wherein the molar ratio of the acrylamide monomer to PSS is 6:0.5, 6:1, or 6:2.

12. The method of claim 10, wherein the PSS has a matrix rigidity of about 54 kPa, about 138 kPa, or about 344 kPa.

13. The method of claim 8, further comprising copolymerizing the acrylamide monomer and the ionic monomer with a bisacrylamide.

14. A method for synthesizing a hydrogel comprising dissolving an acrylamide monomer and an ionic monomer in deionized water, and polymerizing the acrylamide monomer and ionic monomer using a crosslinker and a redox initiator.

15. The method of claim 14, wherein the crosslinker is bisacrylamide.

16. The method of claim 14, wherein the ionic monomer is sodium 4-vinylbenzenesulfonate.

17. A method for expanding stem cells comprising seeding a stem cell onto a synthetic hydrogel, wherein the synthetic hydrogel comprises a heparin mimetic moiety and an acrylamide monomer.

18. The method of claim 17, wherein the heparin mimetic moiety is poly(sodium-4-styrenesulfonate) (PSS).

19. The method of claim 17, wherein the molar ratio of the acrylamide monomer to PSS is 6:0.5, 6:1, or 6:2.

20. The method of claim 17, wherein the stem cell is selected from the group consisting of HUES9, HUES9-Oct4-GFP, HUES6, and human induced pluripotent stem cells (hiPSCs).
